(19) United States
(12) Patent Application Publication
(10) Pub. No.: US 2007/0142414 A1
(43) Pub. Date: Jun. 21, 2007

(54) N-substituted pyrrolopyridinones active as kinase inhibitors

(75) Inventors: Ermes Vanotti, Milano (IT); Maria Menichincheri, MI (IT); Paolo Orsini, Legnano, MI (IT); Alessandra Scolaro, Bresso, MI (IT); Mario Varasi, Milano (IT)

Correspondence Address: SCULLY SCOTT MURPHY & PRESSER, PC, 400 GARDEN CITY PLAZA, SUITE 300, GARDEN CITY, NY, 11530, US

(73) Assignee: Pharmacia Italia S.p.A., Milan (IT)

(21) Appl. No.: 11/303,114

(22) Filed: Dec. 16, 2005

(51) Int. Cl. A61K 31/506; A61K 31/4745; C07D 471/02

(52) U.S. Cl. 514/275; 514/300; 544/331; 546/113

(57) ABSTRACT

Compounds represented by formula (I) 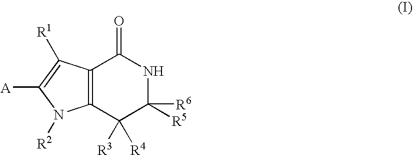
wherein A, R¹, R², R³, R⁴, R⁵, and R⁶ are as defined in the specification, pharmaceutical compositions thereof, and methods of use thereof.

### FIELD OF THE INVENTION

The present invention relates to N-substituted pyrrolopyridinones, to pharmaceutical compositions comprising them and to their use as therapeutic agents, particularly in the treatment of cancer and cell proliferation disorders.

### BACKGROUND OF THE INVENTION

The malfunctioning of protein kinases (PKs) is the hallmark of numerous diseases. A large share of the oncogenes and proto-oncogenes involved in human cancers code for PKs. The enhanced activities of PKs are also implicated in many non-malignant diseases, such as benign prostate hyperplasia, familial adenomatosis, polyposis, neuro-fibromatosis, psoriasis, vascular smooth cell proliferation associated with atherosclerosis, pulmonary fibrosis, arthritis glomerulonephritis and post-surgical stenosis and restenosis. PKs are also implicated in inflammatory conditions and in the multiplication of viruses and parasites. PKs can also play a major role in the pathogenesis and development of neurodegenerative disorders. PKs malfunctioning and disregulation are further discussed in Current Opinion in Chemical Biology 1999, 3, 459-465.

Certain heterocyclic compounds are known in the art as protein kinase inhibitors.

Among them are, for instance, pyrrolo-pyrazoles disclosed in WO 02112242; tetrahydroindazoles disclosed in WO 00/69846; pyrrolo-pyridines disclosed in WO 01/98299; aminophthalazinones disclosed in WO 03/014090 and aminoindazoles disclosed in WO 03/028720.

In addition, pyrrolopyridinone derivatives for the treatment of obesity are disclosed in the patent WO 2003/027114 to Bayer Pharmaceuticals Corporation. In particular a pyridylpyrrolopyridinone, namely 5-cyclohexyl-1-(2,4-dichloro-phenyl)-3-methyl-2-pyridin-3-yl-1,5,6,7-tetrahydro-pyrrolo[3,2-c]pyridin-4-one is reported.

More interestingly, pyrrolopyridinone derivatives, endowed with mitogen activated protein kinase-activated protein kinase-2 inhibitory activity, are disclosed in the patent WO 2004/058762 A1 to Pharmacia Corp. Among the compounds disclosed are, in particular, pyridylpyrrolopyridinones which are mostly substituted by aryl- or aryl-alkenyl-groups at the pyridine moiety;pyridylpyrrolopyridinones being substituted by amino groups or halogen atoms at this same pyridine ring are also therein disclosed as synthetic intermediates. Among these compounds there are, for instance, pyridylpyrrolopyridinones that are alkylated at the nitrogen atom of the pyrrole moiety, such as N-methyl, N-butyl, N-but-3-enyl, N-benzyl, N-(4-methoxycarbonyl)benzyl, N-(4-carboxy)benzyl, N-(4-carbamoyl)benzyl, N-(4-methyl)benzyl, N-(2,4-difluoro)benzyl, N-(4-(2-hydroxyethyl)-carbamoyl)benzyl, N-(3-phenyl)propyl, N-(2-cyclohexyl)ethyl, N-(3-methyl)butyl, N-(3-amino)propyl, N-(2-hydroxy)ethyl, N-acetyl, N-carbamoyl, N-acetoxyacetyl, N-terbutoxycarbonyl and N-(2-trimethylsilanyl-ethoxymethyl) analogs, and a few 2-substituted(halo,aryl)pyrimidylpyrrolopyridinones, such as N-methyl, N-(2-trimethylsilanyl-ethoxymethyl) analogs.

Among the several protein kinases known in the art as being implicated in the growth of cancer cells is Cdc7, an evolutionary conserved serine-threonine kinase which plays a pivotal role in linking cell cycle regulation to genome duplication, being essential for the firing of DNA replication origins (see Montagnoli A. et al., EMBO Journal, Vol. 21, No. 12, pp. 3171-3181, 2002; Montagnoli A. et al., Cancer Research Vol. 64, October 1, pp. 7110-7116, 2004).

### SUMMARY OF THE INVENTION

The invention relates to novel compounds which are useful, in therapy, as agents against a host of diseases caused by and/or associated to a disregulated protein kinase activity and, more particularly, Cdk2 and Cdc7 activity.

The invention also relates to compounds which have protein kinase inhibiting activity and, more particularly, Cdk2 and Cdc7 inhibiting activity.

One aspect of the invention relates to N-substituted pyrrolopyridinone derivative which is represented by formula (I) 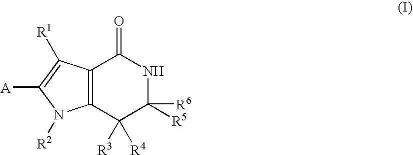
wherein
A is selected from the group consisting of pyridin-4-yl, 3-fluoro-pyridin-4-yl, and 2-amino-pyrimidin-4-yl;
R¹ is selected from the group consisting of hydrogen, halogen and (C₁-C₆)alkyl group;
R² is selected from the group consisting of (C₁-C₆)alkyl, (C₁-C₆)alkenyl, (C₁-C₆)alkynyl, (C₃-C₆)cycloalkyl, (C₁-C₆)haloalkyl, (C₁-C₆)polyfluorinated alkyl, heterocyclyl, aryl, heteroaryl, (C₃-C₆)cycloalkyl-(C₁-C₆)alkyl, heterocyclyl-(C₁-C₆)alkyl, aryl-(C₁-C₆)alkyl, heteroaryl-(C₁-C₆)alkyl, (C₁-C₈)hydroxyalkyl, (C₁-C₈)alkoxy-(C₁-C₈)alkyl, aryloxy-(C₁-C₈)alkyl, heteroaryloxy-(C₁-C₈)alkyl, (C₁-C₈)aminoalkyl, (C₁-C₈)alkylamino-(C₁-C₈)alkyl, (C₁-C₈)dialkylamino-(C₁-C₈)alkyl, carbamoyl(C₁-C₈)alkyl, and alkoxycarbonyl, wherein each of said aryl, heteroaryl, heterocyclyl, aryloxy, or heteroaryloxy moieties can be unsubstituted or substituted by one or substituents, each substituent being independently selected from the group consisting of alkyl, aryl, —OCF₃, —OC(O)alkyl, —OC(O)aryl, —CF₃, heteroaryl, aralkyl, alkylaryl, heteroaralkyl, alkylheteroaryl, hydroxy, hydroxyalkyl, alkoxy, aryloxy, aralkoxy, acyl, aryl, halo, haloalkyl, haloalkoxy, nitro, cyano, carboxy, alkoxycarbonyl, aryloxycarbonyl, aralkoxycarbonyl, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, cycloalkyl, heterocyclyl, heterocyclenyl, —NH(alkyl), —NH(cycloalkyl), and —N(alkyl)₂;
R³, R⁴, R⁵ and R⁶ are each independently selected from the group consisting of hydrogen, (C₁-C₆)alkyl, (C₃-C₆)cycloalkyl, heterocyclyl, aryl, cyloalkyl-(C₁-C₆)alkyl, heterocyclyl-(C₁-C₆)alkyl and aryl-(C₁-C₆)alkyl, wherein each of said aryl or heterocyclyl moieties can be unsubstituted or substituted by one or substituents, each being independently selected from the group consisting of alkyl, aryl, —OCF₃, —OC(O)alkyl, —OC(O)aryl, —CF₃, heteroaryl, aralkyl, alkylaryl, heteroaralkyl, alkylheteroaryl, hydroxy, hydroxyalkyl, alkoxy, aryloxy, aralkoxy, acyl, aryl, halo, haloalkyl, haloalkoxy, nitro, cyano, carboxy, alkoxycarbonyl, aryloxycarbonyl, aralkoxycarbonyl, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, cycloalkyl, heterocyclyl, heterocyclenyl, —NH(alkyl), —NH(cycloalkyl), and —N(alkyl)₂; or
R³ and R⁴ or R⁵ and R⁶, taken together, form a (C₃-C₆)cycloalkyl;
or a pharmaceutically acceptable salt or solvate thereof,
provided that the compound is not:
1-butyl-2-pyridin-4-yl-1,5,6,7-tetrahydro-pyrrolo[3,2-c]pyridin-4-one;
1-benzyl-2-pyridin-4-yl-1,5,6,7-tetrahydro-pyrrolo[3,2-c]pyridin-4-one;
1-but-3-enyl-2-pyridin-4-yl-1,5,6,7-tetrahydro-pyrrolo[3,2-c]pyridin-4-one;
1-(3-methyl-butyl)-2-pyridin-4-yl-1,5,6,7-tetrahydro-pyrrolo[3,2-c]pyridin-4-one;
1-(3-phenyl-propyl)-2-pyridin-4-yl-1,5,6,7-tetrahydro-pyrrolo[3,2-c]pyridin-4-one;
or 1-(2-cyclohexyl-ethyl)-2-pyridin-4-yl-1,5,6,7-tetrahydro-pyrrolo[3,2-c]pyridin-4-one.

Another aspect of the invention relates to a method for treating cell proliferative disorders or conditions, that can be caused by and/or associated with an altered protein kinase activity, by administering to a mammal in need of said treatment an amount of a N-substituted pyrrolopyridinone derivative represented by formula (I) 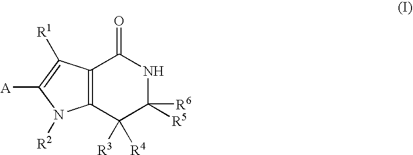
wherein
A is selected from the group consisting of pyridin-4-yl, 3-fluoro-pyridin-4-yl, and 2-amino-pyrimidin-4-yl;
R¹ is selected from the group consisting of hydrogen, halogen and (C₁-C₆)alkyl group;
R² is selected from the group consisting of (C₁-C₆)alkyl, (C₁-C₆)alkenyl, (C₁-C₆)alkynyl, (C₃-C₆)cycloalkyl, (C₁-C₆)haloalkyl, (C₁-C₆)polyfluorinated alkyl, heterocyclyl, aryl, heteroaryl, (C₃-C₆)cycloalkyl-(C₁-C₆)alkyl, heterocyclyl-(C₁-C₆)alkyl, aryl-(C₁-C₆)alkyl, heteroaryl-(C₁-C₆)alkyl, (C₁-C₈)hydroxyalkyl, (C₁-C₈)alkoxy-(C₁-C₈)alkyl, aryloxy-(C₁-C₈)alkyl, heteroaryloxy-(C₁-C₈)alkyl, (C₁-C₈)aminoalkyl, (C₁-C₈)alkylamino-(C₁-C₈)alkyl, (C₁-C₈)dialkylamino-(C₁-C₈)alkyl, carbamoyl(C₁-C₈)alkyl, and alkoxycarbonyl, wherein each of said aryl, heteroaryl, heterocyclyl, aryloxy, or heteroaryloxy moieties can be unsubstituted or substituted by one or substituents, each substituent being independently selected from the group consisting of alkyl, aryl, —OCF₃, —OC(O)alkyl, —OC(O)aryl, —CF₃, heteroaryl, aralkyl, alkylaryl, heteroaralkyl, alkylheteroaryl, hydroxy, hydroxyalkyl, alkoxy, aryloxy, aralkoxy, acyl, aryl, halo, haloalkyl, haloalkoxy, nitro, cyano, carboxy, alkoxycarbonyl, aryloxycarbonyl, aralkoxycarbonyl, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, cycloalkyl, heterocyclyl, heterocyclenyl, —NH(alkyl), —NH(cycloalkyl), and —N(alkyl)₂;
R³, R⁴, R⁵ and R⁶ are each independently selected from the group consisting of hydrogen, (C₁-C₆)alkyl, (C₃-C₆)cycloalkyl, heterocyclyl, aryl, cyloalkyl-(C₁-C₆)alkyl, heterocyclyl-(C₁-C₆)alkyl and aryl-(C₁-C₆)alkyl, wherein each of said aryl or heterocyclyl moieties can be unsubstituted or substituted by one or substituents, each being independently selected from the group consisting of alkyl, aryl, —OCF₃, —OC(O)alkyl, —OC(O)aryl, —CF₃, heteroaryl, aralkyl, alkylaryl, heteroaralkyl, alkylheteroaryl, hydroxy, hydroxyalkyl, alkoxy, aryloxy, aralkoxy, acyl, aryl, halo, haloalkyl, haloalkoxy, nitro, cyano, carboxy, alkoxycarbonyl, aryloxycarbonyl, aralkoxycarbonyl, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, cycloalkyl, heterocyclyl, heterocyclenyl, —NH(alkyl), —NH(cycloalkyl), and —N(alkyl)₂; or
R³ and R⁴ or R⁵ and R⁶, taken together, form a (C₃-C₆)cycloalkyl;
or a pharmaceutically acceptable salt or solvate thereof.

Another aspect of the invention relates to a method of treating cell proliferative disorders caused by and/or associated with an altered Cdc7 kinase activity.

Another aspect of the invention relates to a method of antagonizing activity toward toward Cdk2 or Cdc7, comprising administering to said Cdk2 dor Cdc7 an amount of a compound of Formula (I) that is effective in antagonizing activity toward Cdk2 or Cdc7.

Another aspect of the invention relates to a method of treating a disorder or condition in a mammal, wherein antagonist activity toward toward Cdk2 or Cdc7 is needed in said mammal, comprising administering to said mammal an amount of a compound of Formula (I) that is effective in antagonizing activity toward Cdk2 or Cdc7.

Another aspect of the invention relates to a method of treating a disorder or condition in a mammal for which antagonist activity toward toward Cdk2 or Cdc7 is needed in said mammal, comprising administering to said mammal an amount of a compound of Formula (I) that is effective in treating said disorder or condition.

Another aspect of the invention relates to a method of treating a disorder or condition selected from the group consisting of squamous cell carcinoma, hematopoietic tumors of myeloid or lymphoid lineage, tumors of mesenchymal origin, tumors of the central and peripheral nervous system, melanoma, seminoma, teratocarcinoma, osteosarcoma, xeroderma pigmentosum, keratoxanthoma, thyroid follicular cancer, and Kaposi's sarcoma, in a mammal, comprising administering to said mammal in need of said treatment an amount of a compound of Formula (I) that is effective in treating said condition or disorder.

Another aspect of the invention relates to a method of treating a disorder or condition selected from the group consisting of benign prostate hyperplasia, familial adenomatosis, polyposis, neuro-fibromatosis, psoriasis, vascular smooth cell proliferation associated with atherosclerosis, pulmonary fibrosis, arthritis glomerulonephritis, post-surgical stenosis and restenosis, in a mammal, comprising administering to said mammal in need of said treatment an amount of a compound of Formula (I) that is effective in treating said condition or disorder.

Another aspect of the invention relates to a method of treating a disorder or condition selected from the group consisting of carcinoma, squamous cell carcinoma, hematopoietic tumors of myeloid or lymphoid lineage, tumors of mesenchymal origin, tumors of the central and peripheral nervous system, melanoma, seminoma, teratocarcinoma, osteosarcoma, xeroderma pigmentosum, keratoxanthoma, thyroid follicular cancer, and Kaposi's sarcoma, in a mammal, comprising administering to said mammal in need of said treatment an amount of a compound of Formula (I) that is effective in antagonizing activity toward toward Cdk2 or Cdc7.

Another aspect of the invention relates to a method of treating a disorder or condition selected from the group consisting of benign prostate hyperplasia, familial adenomatosis, polyposis, neuro-fibromatosis, psoriasis, vascular smooth cell proliferation associated with atherosclerosis, pulmonary fibrosis, arthritis glomerulonephritis, post-surgical stenosis and restenosis, in a mammal, comprising administering to said mammal in need of said treatment an amount of a compound of Formula (I) that is effective in antagonizing activity toward toward Cdk2 or Cdc7.

Another aspect of the invention relates to a method of treating a disorder or condition selected from the group consisting of squamous cell carcinoma, leukemia, acute lymphocitic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma, acute and chronic myelogenous leukemias, myelodysplastic syndrome, promyelocytic leukemia, fibrosarcoma, rhabdomyosarcoma, astrocytoma, neuroblastoma, glioma, schwannomas, melanoma, seminoma, teratocarcinoma, osteosarcoma, xeroderma pigmentosum, keratoxanthoma, thyroid follicular cancer, Kaposi's sarcoma and carcinoma of the bladder, breast, colon, kidney, liver, lung, esophagus, gall-bladder, ovary, pancreas, stomach, cervix, thyroid, prostate, or skin, in a mammal, comprising administering to said mammal in need of said treatment an amount of a compound of Formula (I) that is effective in treating said condition or disorder.

Another aspect of the invention relates to a method of treating a disorder or condition selected from the group consisting of squamous cell carcinoma, leukemia, acute lymphocitic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma, acute and chronic myelogenous leukemias, myelodysplastic syndrome, promyelocytic leukemia, fibrosarcoma, rhabdomyosarcoma, astrocytoma, neuroblastoma, glioma, schwannomas, melanoma, seminoma, teratocarcinoma, osteosarcoma, xeroderma pigmentosum, keratoxanthoma, thyroid follicular cancer, Kaposi's sarcoma and carcinoma of the bladder, breast, colon, kidney, liver, lung, esophagus, gall-bladder, ovary, pancreas, stomach, cervix, thyroid, prostate, or skin, in a mammal, comprising administering to said mammal in need of said treatment an amount of a compound of Formula (I) that is effective in antagonizing activity toward toward Cdk2 or Cdc7.

In other embodiments, the above mentioned methods exclude the following compounds from the the compound of Formula (I):
1-butyl-2-pyridin-4-yl-1,5,6,7-tetrahydro-pyrrolo[3,2-c]pyridin-4-one;
1-benzyl-2-pyridin-4-yl-1,5,6,7-tetrahydro-pyrrolo[3,2-c]pyridin-4-one;
1-but-3-enyl-2-pyridin-4-yl-1,5,6,7-tetrahydro-pyrrolo[3,2-c]pyridin-4-one;
1-(3-methyl-butyl)-2-pyridin-4-yl-1,5,6,7-tetrahydro-pyrrolo[3,2-c]pyridin-4-one;
1-(3-phenyl-propyl)-2-pyridin-4-yl-1,5,6,7-tetrahydro-pyrrolo[3,2-c]pyridin-4-one;
and 1-(2-cyclohexyl-ethyl)-2-pyridin-4-yl-1,5,6,7-tetrahydro-pyrrolo[3,2-c]pyridin-4-one.

Another aspect of the invention relates to a pharmaceutical composition comprising an amount of the compound of Formula (I), or a pharmaceutically acceptable salt or solvate thereof, and a pharmaceutically acceptable carrier.

A more complete appreciation of the invention, and many of the attendant advantages thereof, will be readily understood as the same becomes better understood by reference to the following detailed description.

### DETAILED DESCRIPTION OF THE INVENTION

The compounds of formula (I) of the invention can have asymmetric carbon atoms and can therefore exist as individual optical isomers, as racemic admixtures or as any other admixture including a majority of one of the two optical isomers, which are all to be intended as comprised within the scope of the present invention.

Likewise, the use as an antitumor agent of all the possible isomers and their admixtures and of both the metabolites and the pharmaceutically acceptable bio-precursors (otherwise referred to as pro-drugs) of the compounds of formula (I) are also within the scope of the present invention. Prodrugs are any covalently bonded compounds which release the active parent drug, according to formula (I), in vivo.

In cases when compounds can exist in tautomeric forms, for instance keto-enol tautomers, each tautomeric form is contemplated as being included within this invention whether existing in equilibrium or predominantly in one form.

Except where stated otherwise, the following definitions apply throughout the present specification and claims. These definitions apply regardless of whether a term is used by itself or in combination with other terms. Hence the definition of “alkyl” applies to “alkyl” as well as to the “alkyl” portions of “alkylamino”, “dialkylamino” etc.

As used above, and throughout the specification, the following terms, unless otherwise indicated, shall be understood to have the following meanings:

“Mammal” means humans and other animals.

“Treating” refers to, and includes, reversing, alleviating, inhibiting the progress of, or preventing, a disease, disorder or condition, or one or more symptoms thereof; and, “treatment” and “therapeutically” refer to the act of treating, as defined above.

The term “effective amount” means an amount of compound of the present invention that is capable of treating a specific disease or antagonizing a specific enzyme, such as a specific protein kinase. The particular dose of compound administered according to the invention will be determined by the particular circumstances surrounding the case including, for example, the compound administered, the route of administration, the state of being of the subject, and the severity of the pathological condition being treated.

“Alkyl” means an aliphatic hydrocarbon group, which can be straight or branched. Branched means that one or more lower alkyl groups such as methyl, ethyl or propyl, are attached to a linear alkyl chain. The alkyl group can be substituted by one or more substituents which can each be independently selected from the group consisting of halo, alkyl, aryl, cycloalkyl, cyano, hydroxy, alkoxy, alkylthio, amino, —NH(alkyl), —NH(cycloalkyl), —N(alkyl)₂, carboxy and —C(O)O-alkyl. Non-limiting examples of suitable alkyl groups include methyl, ethyl, n-propyl, isopropyl, n-butyl, and t-butyl, isobutyl, sec-butyl, n-pentyl, n-hexyl, and the like.

“Alkenyl” means an aliphatic hydrocarbon group containing at least one carbon-carbon double bond and which can be straight or branched. The term “substituted alkenyl” means that the alkenyl group can be substituted by one or more substituents which can be the same or different, each substituent being independently selected from the group consisting of halo, alkyl, aryl, cycloalkyl, cyano, and alkoxy. Non-limiting examples of suitable alkenyl groups include ethenyl, propenyl, and n-butenyl.

“Alkynyl” means an aliphatic hydrocarbon group containing at least one carbon-carbon triple bond and which can be straight or branched. Branched means that one or more lower alkyl groups such as methyl, ethyl or propyl, are attached to a linear alkynyl chain. Non-limiting examples of suitable alkynyl groups include ethynyl, propynyl, and 2-butynyl. The term “substituted alkynyl” means that the alkynyl group can be substituted by one or more substituents each being independently selected from the group consisting of alkyl, aryl and cycloalkyl.

“amino” means an —NH₂ group whilst the term arylamino comprises any group —NH-aryl, wherein aryl is as defined below.

“halogen” means a fluorine, chlorine, bromine or iodine atom.

“polyfluorinated alkyl” means any alkyl group as defined above being substituted by two or more fluorine atoms such as, for instance, trifluoromethyl, 2,2,2-trifluoroethyl, 3,3,3-trifluoropropyl, 1,1-difluoroethyl, 3,3-difluoropropyl and the like.

With the term aryl, the present invention contemplates any carbocyclic or heterocyclic hydrocarbon with from 1 to 2 ring moieties, either fused or linked to each other by single bonds, wherein at least one of the rings is aromatic. If present, any aromatic heterocyclic hydrocarbon also referred to as heteroaryl group, comprises a 5 to 6 membered ring with from 1 to 3 heteroatoms selected among N, O or S.

The aryl group can be unsubstituted or substituted on the ring with one or more substituents, each being independently selected from the group consisting of alkyl, aryl, OCF₃, OCOalkyl, OCOaryl, CF₃, heteroaryl, aralkyl, alkylaryl, heteroaralkyl, alkylheteroaryl, hydroxy, hydroxyalkyl, alkoxy, aryloxy, aralkoxy, acyl, aryl, halo, haloalkyl, haloalkoxy, nitro, cyano, carboxy, alkoxycarbonyl, aryloxycarbonyl, aralkoxycarbonyl, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, cycloalkyl, heterocyclyl, heterocyclenyl, —NH(alkyl), —NH(cycloalkyl), and —N(alkyl)₂. Non-limiting examples of suitable aryl groups include phenyl and naphthyl. The “aryl” group can also be substituted by linking two adjacent carbons on its aromatic ring via a combination of one or more carbon atoms and one or more oxygen atoms such as, for example, methylenedioxy, ethylenedioxy, and the like. Examples of aryl groups according to the invention are, for instance, phenyl, biphenyl, α- or β-naphthyl, dihydronaphthyl, thienyl, benzothienyl, furyl, benzofuranyl, pyrrolyl, imidazolyl, pyrazolyl, thiazolyl, isothiazolyl, oxazolyl, isoxazolyl, pyridyl, pyrazinyl, pyrimidinyl, pyridazinyl, indolyl, isoindolyl, purinyl, quinolyl, isoquinolyl, dihydroquinolinyl, quinoxalinyl, benzodioxolyl, indanyl, indenyl, triazolyl, and the like.

“cycloalkyl” means a non-aromatic mono- or multicyclic ring system. The cycloalkyl can be optionally substituted on the ring by replacing an available hydrogen on the ring by one or more substituents, each being independently selected from the group consisting of alkyl, aryl, heteroaryl, aralkyl, alkylaryl, aralkenyl, heteroaralkyl, alkylheteroaryl, heteroaralkenyl, hydroxy, hydroxyalkyl, alkoxy, aryloxy, aralkoxy, acyl, aryl, halo, nitro, cyano, carboxy, alkoxycarbonyl, aryloxycarbonyl, aralkoxycarbonyl, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, cycloalkyl, cycloalkenyl, heterocyclyl, heterocyclenyl, —NH(alkyl), —NH(cycloalkyl), and —N(alkyl)₂ Non-limiting examples of suitable monocyclic cycloalkyls include cyclopropyl, cyclobutyl, cyclopentyl and cyclohexyl.

“heterocyclyl” means any 5 or 6 membered heterocyclic ring comprising from 1 to 3 heteroatoms selected among N, O or S. If the said heterocycle or heterocyclyl group is an aromatic heterocycle, also referred to as heteroaryl, it is encompassed by the above definition given to aryl groups.

As such, besides the above aromatic heterocycles, the term heterocyclyl also encompasses saturated or partially unsaturated heterocycles such as, for instance, pyrroline, pyrrolidine, imidazoline, imidazolidine, pyrazoline, pyrazolidine, piperidine, piperazine, morpholine, and the like.

When the aryl or heteroaryl group is optionally substituted, the substituents are preferably selected from alkyl, haloalkyl, polyfluoroalkyl, hydroxyalkyl, aminoalkyl, amino, alkylamino, dialkylamino, cyano, hydroxy, alkoxy, halogen, as herein defined.

Pharmaceutically acceptable salts of the compounds of formula (I) include the acid addition salts with inorganic or organic acids such as, for instance, nitric, hydrochloric, hydrobromic, sulfuric, perchloric, phosphoric, acetic, trifluoroacetic, propionic, glycolic, lactic, oxalic, malonic, malic, maleic, tartaric, citric, benzoic, cinnamic, mandelic, methanesulphonic, isethionic and salicylic acid.

Prodrugs and solvates of the compounds of the invention are also contemplated herein. The term “prodrug”, as employed herein, denotes a compound that is a drug precursor, which, upon administration to a subject, undergoes chemical conversion by metabolic or chemical processes to yield a compound of formula (I) or a salt and/or solvate thereof. A discussion of prodrugs is provided in T. Higuchi and V. Stella, Pro-drugs as Novel Delivery Systems (1987) Volume 14 of the A.C.S. Symposium Series, and in Bioreversible Carriers in Drug Design, (1987) Edward B. Roche, ed., American Pharmaceutical Association and Pergamon Press, both of which are incorporated herein by reference thereto.

“Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

When any variable (e.g., aryl, alkyl, etc.) occurs more than one time in any constituent or in Formula (I), its definition on each occurrence is independent of its definition at every other occurrence. Also, combinations of substituents and/or variables are permissible only if such combinations result in stable compounds.

Except where stated otherwise, the following definitions apply throughout the present specification and claims. These definitions apply regardless of whether a term is used by itself or in combination with other terms. Hence the definition of “alkyl” applies to “alkyl” as well as to the “alkyl” portions of “alkylamino”, “dialkylamino” etc.

A preferred class of compounds is represented by the derivatives of formula (I) wherein A is as defined above, R¹, R², R⁵ and R⁶ are as defined above and both R³ and R⁴ are hydrogen atoms.

Another preferred class of compounds is represented by the derivatives of formula (I) wherein A is as defined above, R¹, R², R³ and R⁴ are as defined above and both R⁵ and R⁶ are hydrogen atoms.

Still more preferred compounds of the invention, within the above classes, are the derivatives of formula (I) wherein A is a 2-amino-pyrimidin-4-yl group, R¹ is a hydrogen atom and R², R³, R⁴, R⁵, R⁶ are as defined above.

The compounds of formula (I) and the pharmaceutically acceptable salts thereof can be obtained by different general procedures, for instance, by N-derivatization of the pyrrole nitrogen atom of compounds of formula (I), where R² is hydrogen, by reacting them with electrophiles bearing groups such as, for example, halides, triflates, mesylates, tosylates and the like, so that a compound with R² as defined is obtained, or by direct construction of (I), where R² is as defined, from simpler constituents, for instance via a Hantzsch type reaction.

The following schemes, which are described hereinbelow, further depict how to obtain the compounds of formula (I):

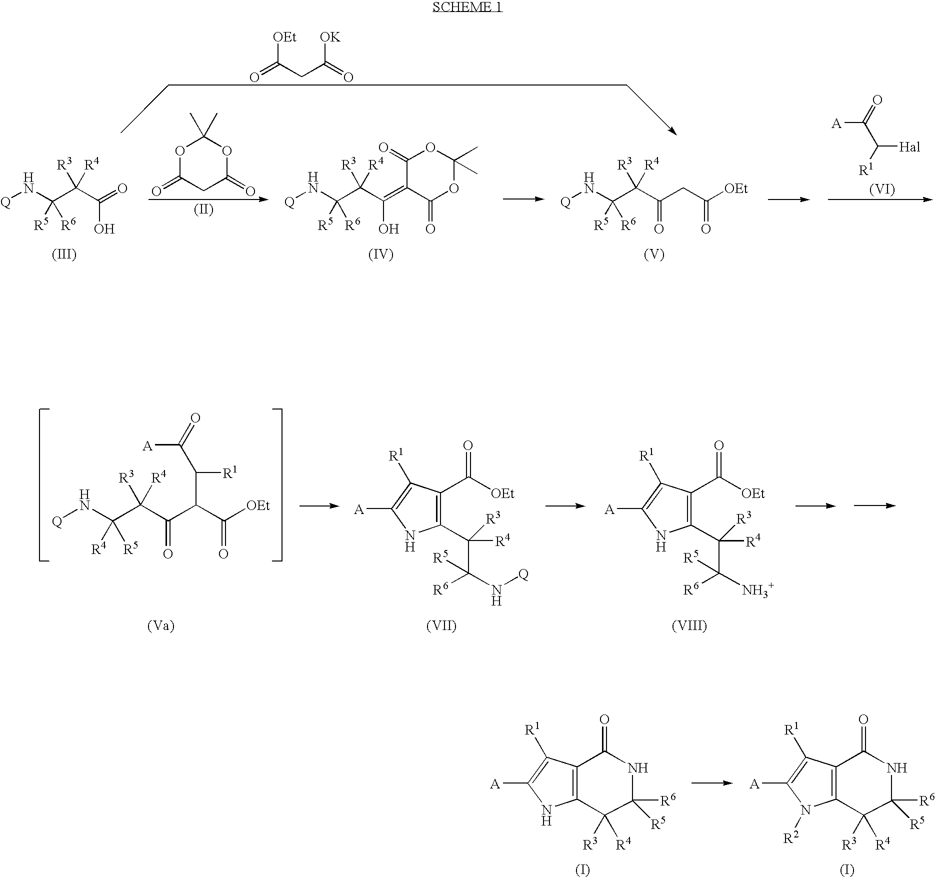

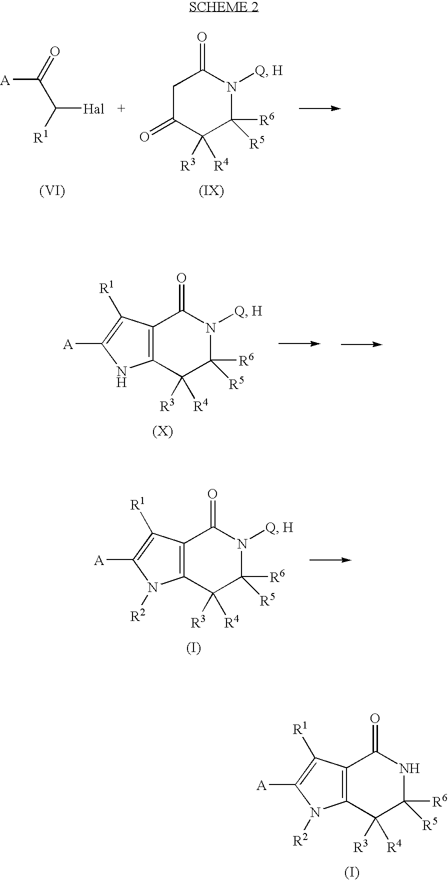

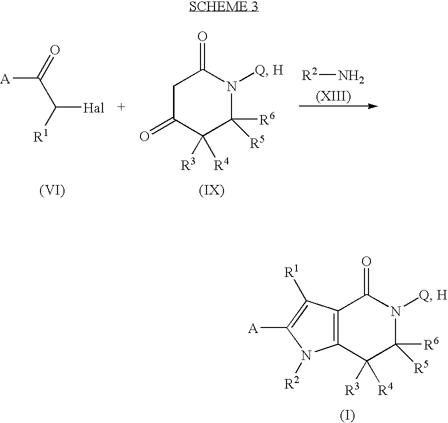

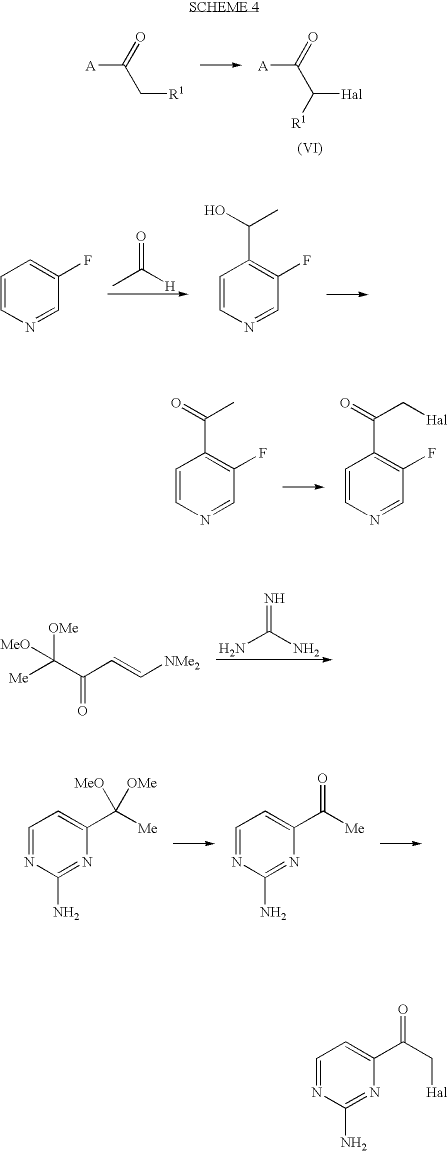

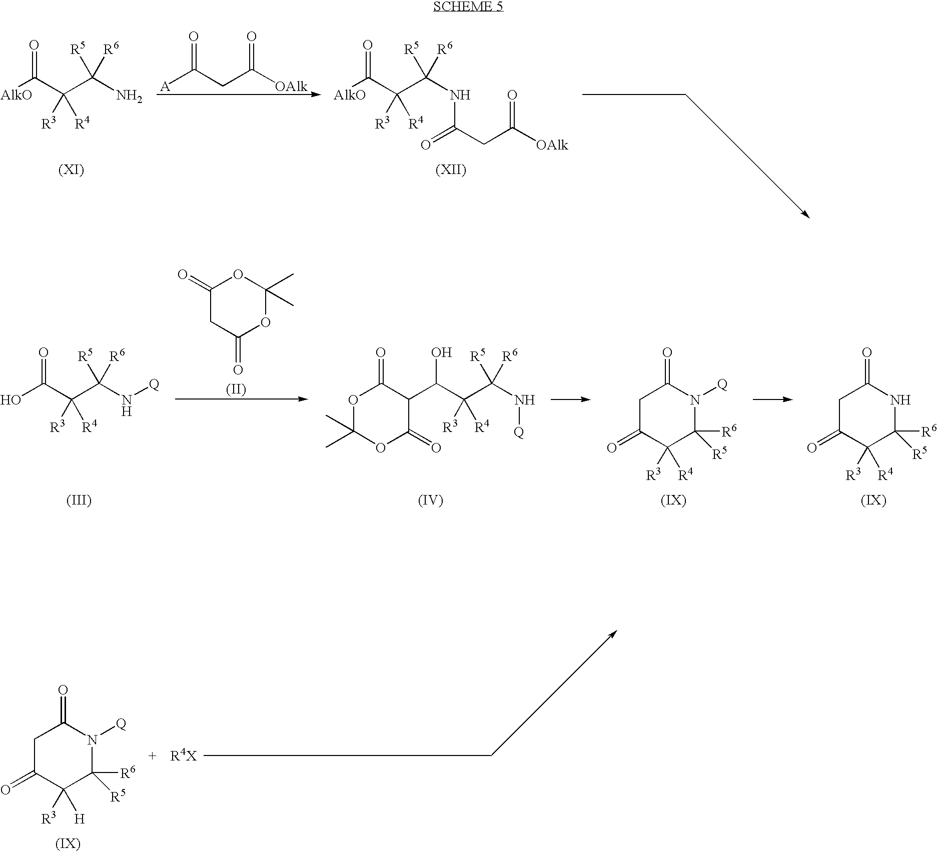

The compounds of formula (I) where R² is a hydrogen atom can be obtained by a process comprising:
a) reacting the Meldrum's acid of formula (II) with a suitable aminoacid derivative of formula (III) so as to obtain a compound of formula (IV) 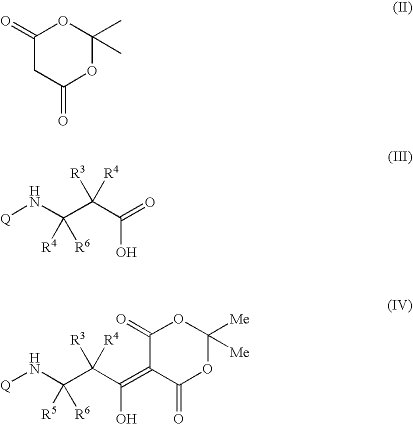
wherein Q is a suitable nitrogen protecting group, most preferably a t-butoxycarbonyl (Boc), and R³, R⁴, R⁵ and R⁶ are as above defined;
b) heating the compound of formula (IV) in the presence of ethanol so as to obtain a compound of formula (V), 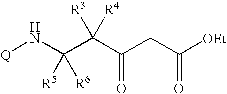
c) reacting the compound of formula (V) with a derivative of formula (VI) and treating the resulting intermediate (Va) with ammonium acetate, so as to obtain a compound of formula (VII) 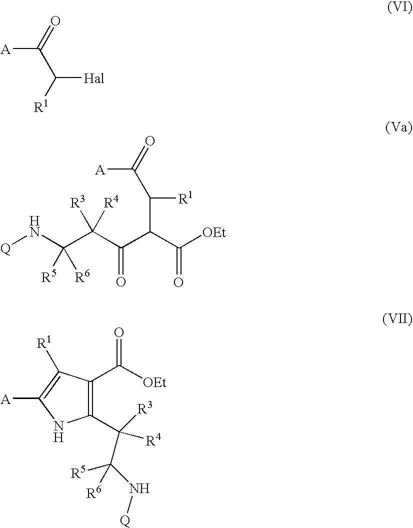
wherein A, R³, R⁴, R⁵, R⁶ are as defined above, R¹ is a hydrogen atom or a straight or branched (C₁-C₆)alkyl group, Q is the aforementioned nitrogen protecting group and Hal represents a suitable halogen atom;
d) deprotecting the compound of formula (VII) by removing the Q group in a suitable manner, e.g. by acidic treatment when Q is t-butoxy carbonyl so as to obtain a compound of formula (VIII) 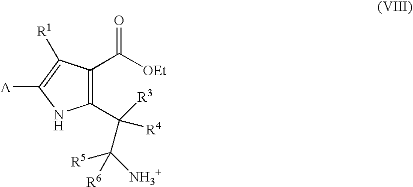
e) refluxing the compound of formula (VIII) in the presence of a base so as to obtain the compound of formula (I, R²═H) and, optionally, converting it into another compound of formula (I, R²═H).

In an alternative embodiment, the product (V) can be synthesized reacting the aminoacid derivative of formula (III) with ethyl potassium malonate in the presence of 1,1′-carbonyldiumidazole and magnesium chloride.

The above process is an analogy process that can be carried out according to methods well known in the art.

According to step (a) of the process, the Meldrum's acid of formula (II) is reacted with the aminoacid derivative of formula (III) in the presence of a base, for instance dimethylaminopyridine (DMAP), and of a suitable solvent such as dichloromethane (DCM). The reaction is carried out in the presence of a carbodiimide such as N,N′-dicyclohexylcarbodiimide at a temperature of about 0° C. and for a time varying from about 2 hours to about 24 hours.

In step (b) of the process, the crude material of formula (IV) obtained in step (a) is dissolved in ethanol and heated at a temperature ranging from about 50° C. to refluxing temperature, for a time of about 2 hours to about 12 hours, thus affording the compound of formula (V).

In the alternative route to obtain the compound of formula (V), the compound of formula (III) is reacted with potassium malonate in the presence of 1,1′-carbonyldiimidazole and magnesium chloride. In this instance, to a solution of (III), in anhydrous tetrahydrofuran (THF), 1,1′-carbonyldiimidazole is added; the solution is left shaking 2 hours and ethyl potassium malonate and magnesium chloride are added. The temperature is then brought to a suitable value, that is, from 30 to 70° C. A preferred temperature is 45° C. Stirring is carried out for a suitable time from 4 to 18 h.

According to step (c) of the process, the compound of formula (V) is reacted with a suitable heteroaryl derivative of formula (VI), in the presence of sodium hydride and, successively, of ammonium acetate, in a suitable solvent such as, for instance, tetrahydrofuran so as to obtain a compound of formula (VII). Preferably, within the compounds of formula (VI), Hal represents a bromine or chlorine atom. In this instance to a solution of (V) in dry THF, sodium hydride is added; stirring is carried out for 30 min and a suitable heteroaryl derivative (VI) is added. The reaction is carried out at a temperature of about 0° C. and for a time varying from about 1 hour to about 6 hours. To the crude material obtained, ammonium acetate in ethanol is added. Stirring at room temperature is carried out for a suitable time from about 5 to about 24 hours.

The obtained compound of formula (VII) is then deprotected at the nitrogen atom, in step (d) of the process, through acidic treatment, so as to obtain the corresponding amino derivative of formula (VIII) in the form of an acid addition salt.

The reaction is carried out according to conventional methods in the presence of a suitable acid such as, for instance, hydrochloric or trifluoroacetic acid and of a suitable solvent, for instance, tetrahydrofuran, dioxane or the like. Stirring at room temperature is maintained for a suitable period of time.

According to step (e) of the process, the compound of formula (VIII) is then converted into the compound of formula (I, R²═H) by treatment with a base, for instance sodium carbonate, in the presence of a lower alcohol such as ethanol. The reaction is carried out at refluxing temperature for a time varying from about 12 hours to about 24-48 hours.

The starting compounds of formulae (II), (III) and (VI), as well as any other reactant(s) of the process, are known or, if not commercially available per se, they can be easily prepared according to known methods starting from known compounds.

As an example, the heteroaryl derivatives of formula (VI) can be prepared by halogenating, e.g. brominating or chlorinating, a suitable heteroaryl-ethanone derivative, according to the following pathway:

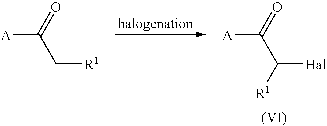

The above reaction occurs by working under conventional methods, for instance in the presence of bromine and in a suitable solvent such as a mixture of acetic and hydrobromic acid, for a time varying between about 1 hour and about 24 hours. Alternatively, a suitably activated heteroaryl derivative, e.g. an enolether or silylether, can be reacted with a halogen source, for instance N-bromo-succinimide (NBS), in a suitable solvent, such as tetrahydrofuran/water mixtures.

Among the suitable heteroaryl-ethanone derivatives subdued to halogenation, the present invention contemplates for instance, 1-pyridin-4-ylethanone, 1-pyridin-4-ylpropan-1-one, 1-(3-fluoropyridin-4-yl)ethanone and 1-(2-aminopyrimidin-4-yl)ethanone. 1-(3-Fluoropyridin-4-yl)ethanone can be prepared, for example, by reacting commercial 3-fluoropyridine with acetaldehyde in the presence of a base, such as, for example, lithiumdiisopropylamide (LDA) and oxidizing the so obtained 1-(3-fluoropyridin-4-yl)ethanol by means of, for instance, manganese dioxide in a suitable solvent, like toluene. 1-(2-aminopyrimidin-4-yl)ethanone can be obtained according to the following path: 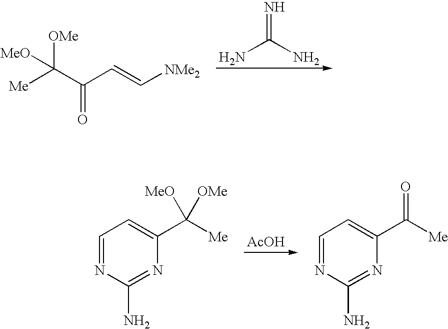
1-(Dimethylamino)-4,4-dimethoxy-1-penten-3-one is a known compound which can be prepared according to known methods, for instance as reported in J. Het. Chem., 22(6), 1723-6, 1985. It is easily reacted with guanidine, for instance being available in the form of an acid addition salt, e.g. as guanidinium hydrochloride salt. The reaction is carried out under basic conditions, for instance in the presence of sodium ethylate and of a suitable solvent such as a lower alcohol, preferably ethanol. The reaction occurs at refluxing temperature for a suitable time up to about 24 hours.

The above reaction results in the production of the intermediate pyrimidine compound which is then converted into the final intermediate through acidic treatment at room temperature, for instance in the presence of acetic acid.

According to an alternative approach, the compounds of formula (I, R²═H) can be also prepared according to the following synthetic scheme, by reacting the above heteroaryl derivative of formula (VI) with a suitable piperidine-dione derivative of formula (IX) wherein Q is H or the aforementioned nitrogen protecting group, preferably tert-butoxycarbonyl or p-methoxybenzyl, p-methoxyethylbenzyl, p-methoxyphenyl group.

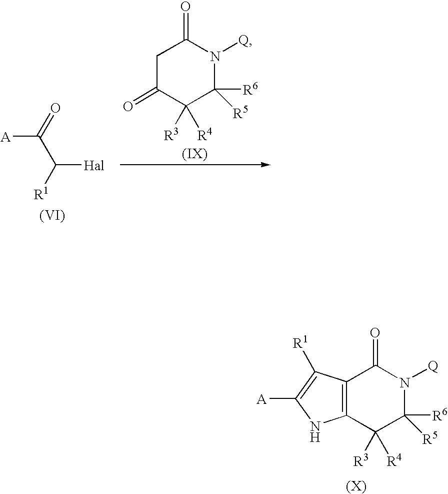

The above reaction occurs in the presence of ammonium acetate and of a suitable solvent such as, for instance, a lower alcohol or acetic acid. Preferably, the reaction is carried out in the presence of ethanol by working at room temperature and for a suitable time varying from about 2 hours to about 24 hours.

Also the piperidine-dione derivative (IX) is a known compound or, alternatively, can be prepared by known methods, for instance according to the synthetic pathway below, wherein “Alk” stands for a suitable lower alkyl group, e.g. propyl, ethyl, methyl, etc., and “A” stands for chloro or —OAlk:

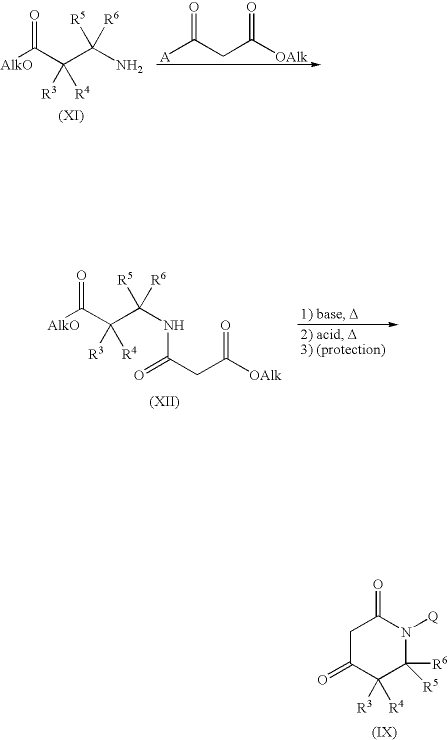

In this respect, a suitable β-amino-carboxyester (XI) derivative wherein R³, R⁴, R⁵ and R⁶ have the above reported meanings, is reacted with dialkylmalonate or, alternatively, with 3-chloro-3-oxopropanoic acid alkyl ester, for instance, dimethylmalonate or ethyl 3-chloro-3-oxopropanoate, respectively. When A is chloro the reaction is carried out under basic conditions, for instance in the presence of triethylamine, and in a suitable solvent such as dichloromethane, at a temperature comprised between room temperature to reflux. When A is —OAlk the reaction is carried out with or without basic conditions and more conveniently in the absence of solvents at reflux temperature of the dialkylmalonate.

When not commercially available, the above-mentioned β-amino-carboxyester derivatives (XI) can be obtained according to well known procedures described in the literature.

The intermediate derivative thus obtained (XII) is then converted into the compound of formula (IX), first by reacting it under basic conditions, e.g. in the presence of sodium methylate and of a suitable solvent, preferably toluene, at refluxing temperature and for a time varying between about 2 hours and about 24 hours. Subsequently, the product of the former step is reacted as such, without being isolated, with an acetonitrile/water/acetic acid mixture under refluxing conditions and for a time varying between about 12 hours and about 24 hours. Optionally the piperidin-dione (IX) can be protected with a suitable protecting group Q.

In the alternative, the piperidine-dione derivative (IX) can be prepared, for instance, according also to the alternative synthetic pathway below:

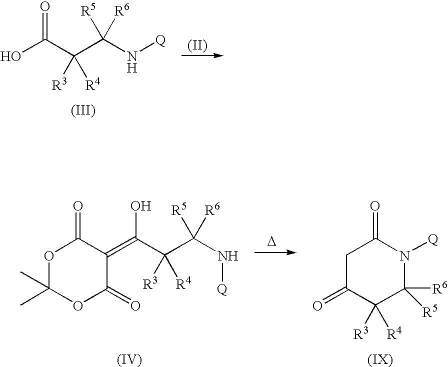

In the procedure the Meldrum's acid of formula (II) is reacted with a suitable aminoacid derivative of formula (III) so as to obtain a compound of formula (IV) wherein Q is a suitable nitrogen protecting group and R³, R⁴, R⁵ and R⁶ are as above defined. The compound of formula (IV) is then cyclized by dissolving it in a suitable solvent, for instance ethylacetate, and refluxing for a period of time from 1 to 24 hours;
or, in the alternative, the piperidine-dione derivative (IX) can be modified according to the synthetic pathway below, wherein Q stands for a suitable nitrogen-protecting group such as, in particular, tert-butoxycarbonyl, or other groups, such as p-methoxybenzyl, p-methoxyethylbenzyl, p-methoxyphenyl, and X is halide, triflate, mesylate, tosylate and the like:

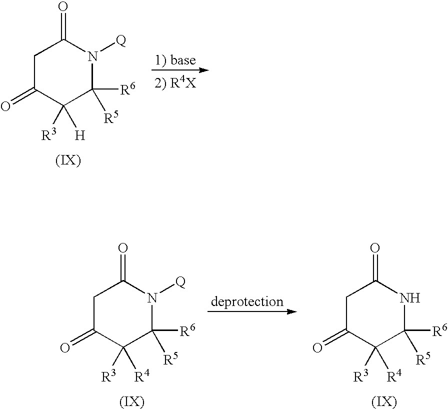

In this respect, a suitable piperidinedione derivative (IX) wherein R³, R⁵ and R⁶ and Q have the above reported meanings, is reacted with a base, for instance lithium bis(trimethylsilyl)amide (LiHMDS). The reaction is carried out in a suitable solvent such as tetrahydrofuran, at a temperature comprised between −78° C. and room temperature. The reaction mixture is then treated with a suitable R⁴X, where X is a group such as halide, triflate, mesylate, tosylate and the like, thus obtaining another compound of formula (IX). The compound thus obtained, where Q is for instance a tert-butoxycarbonyl group, can be converted into another compound of formula (IX) by treating it with acidic conditions, e.g. in the presence of trifluoroacetic acid and of a suitable solvent, preferably dichloromethane, at room temperature and for a time ranging between about 1 hour and about 6 hours.

The final compound of formula (I) thus obtained can be then converted into another compound of formula (I) according to well-known methods in the art. As an example, the compounds of formula (I), wherein R¹ represents a hydrogen atom, can be easily converted into the corresponding compounds wherein R¹ is a halogen atom through conventional methods reported in the literature for the halogenation of pyrrole derivatives.

As indicated above, the compounds of formula (I), where R² is as defined, can be prepared by reacting the pyrrolopyridinones of formula (I), where R² is an hydrogen atom, with a suitable electrophile, such as a convenient halide or a triflate, in a suitable solvent, such as dimethylformamide, THF, dioxane, in the presence of a suitable base, such as sodium hydride, at temperatutes ranging from −30° C. to room temperature, most often at 0° C., for a convenient period of time, from 1 to 24 h.

Alternatively a different base can be used, for instance potassium or cesium carbonate, optionally in the presence of a crown ether, for example 18-crown-6, at temperatures from room temperature to 100° C., optionally in a microwave cavity, in a suitable solvent, such as DMF.

According to an alternative approach, the compounds of formula (I) can be also directly prepared according to the following synthetic scheme, by reacting the above described heteroaryl derivative of formula (VI) with a suitable piperidine-dione derivative of formula (IX) wherein Q is H or the aforementioned nitrogen protecting group, preferably tert-butoxycarbonyl group, in the presence of a suitable amine of formula (XIII), where R² is as defined.

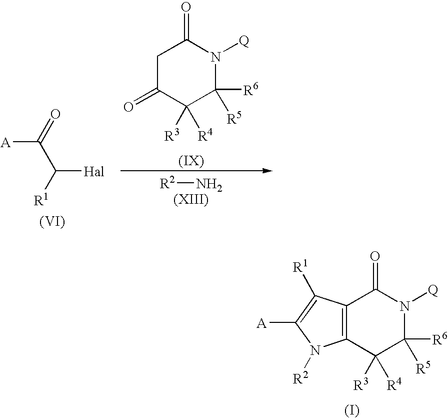

The reaction occurs in the presence of a suitable solvent such as, for instance, a lower alcohol or acetic acid. Preferably, the reaction is carried out in the presence of ethanol by working at temperatures ranging from room temperature to 100° C. and for a suitable time varying from about 2 hours to about 24 hours.

When Q is a protecting group, for instance a tert-butoxycarbonyl group, the desired compound of formula (1), where Q=H, can be obtained by treating it with acidic conditions, e.g. in the presence of trifluoroacetic acid and of a suitable solvent, preferably dichloromethane, at room temperature and for a time comprised between about 1 hours and about 6 hours.

Likewise, the conversion of a compound of formula (I) into a pharmaceutically acceptable salt is easily carried out according to known methods, e.g. by contacting any free base of formula (I) with any suitable pharmaceutically acceptable acid.

From all of the above, it is understood to the skilled person artisan that when preparing the compounds of formula (I) according to the aforementioned processes, comprehensive of any variant thereof, optional functional groups within the starting materials or the intermediates thereof, which could give rise to unwanted side reactions, need to be properly protected according to conventional techniques. Likewise, the conversion of these latter into the free deprotected compounds can be carried out according to known procedures.

By analogy, any compound of formula (I) which is susceptible of being salified can be easily converted into the corresponding acid addition salt, by working in the presence of any pharmaceutically acceptable acid, for instance selected among those previously reported.

As it will be readily appreciated, if the compounds of formula (I) prepared according to the process described above are obtained as a mixture of isomers, their separation into the single isomers of formula (I), according to conventional techniques, is also within the scope of the present invention.

Conventional techniques for racemate resolution include, for instance, partitioned crystallization of diastereoisomeric salt derivatives or preparative chiral HPLC.

Pharmacology

The compounds of formula (I) are active as protein kinase inhibitors and are therefore useful, for instance, to restrict the unregulated proliferation of tumor cells.

In therapy, they can be used in the treatment of various tumors, such as those formerly reported, as well as in the treatment of other cell proliferative disorders such as psoriasis, vascular smooth cell proliferation associated with atherosclerosis and post-surgical stenosis and restenosis and in the treatment of Alzheimer's disease.

The compounds of the invention can be also active as inhibitors of other protein kinases such as, for instance, protein kinase C in different isoforms, Met, PAK-4, PAK-5, ZC-1, STLK-2, DDR-2, Aurora 1, Aurora 2, Bub-1, PLK, Chk1, Chk2, HER2, raf1, MEK1, MAPK, EGF-R, PDGF-R, FGF-R, IGF-R, VEGF-R, PI3K, weel kinase, Src, Abl, AKT, ILK, MK-2, IKK-2, Cdk in different isoforms, Nek, CK2, GSK3, SULU, PKA, PKC, PDK, RET, KIT, LCK, TRKA and thus be effective in the treatment of diseases associated with other protein kinases.

The inhibiting activity of putative Cdc7 inhibitors and the potency of selected compounds is determined through a method of assay based on the use of Dowex resin capture technology.

The assay consists of the transfer of radioactivity labeled phosphate moiety by the kinase to an acceptor substrate. The resulting 33P-labeled product is separated from unreacted tracer, transferred into a scintillation cocktail and light emitted is measured in a scintillation counter.

Inhibition Assay of Cdc7 Activity

The inhibiting activity of putative Cdc7 inhibitors and the potency of selected compounds can be determined through a method of assay based on the use of Dowex resin capture technology.

The assay consists of the transfer of radioactivity labeled phosphate moiety by the kinase to an acceptor substrate. The resulting 33P-labeled product is separated from unreacted tracer, transferred into a scintillation cocktail and light emitted is measured in a scintillation counter.

The inhibition assay of Cdc7/Dbf4 activity is performed according to the following protocol:

The MCM2 substrate is trans-phosphorylated by the Cdc7/Dbf4 complex in the presence of ATP traced with γ³³-ATP. The reaction is stopped by addition of Dowex resin in the presence of formic acid. Dowex resin particles capture unreacted γ³³-ATP and drag it to the bottom of the well while ³³P phosphorylated MCM2 substrate remains in solution. The supernatant is collected, transferred into Optiplate plates and the extent of substrate phosphorylation is evaluated by β counting.

The inhibition assay of Cdc7/Dbf4 activity was performed in 96 wells plate according to the following protocol:

To each well of the plate were added:
10 μl test compound (10 increasing concentrations in the nM to uM range to generate a dose-response curve). The solvent for test compounds contained 3% DMSO. (final concentration 1%)
10 μl substrate MCM2 (6 μM final concentration), a mixture of cold ATP (2 μM final concentration) and radioactive ATP (1/5000 molar ratio with cold ATP).
10 μl enzyme (Cdc7/Dbf4, 2 nM final concentration) that started the reaction. The buffer of the reaction consisted in 50 mM HEPES pH 7.9 containing 15 mM MgCl₂, 2 mM DTT, 3 uM NaVO₃, 2 mM glycerophosphate and 0.2 mg/ml BSA.

After incubation for 60 minutes at room temperature, the reaction was stopped by adding to each well 150 μl of Dowex resin in the presence of 150 mM formic acid. After another 60 min incubation, 50 μL of suspension were withdrawn and transferred into 96-well OPTIPLATEs containing 150 μl of MicroScint 40 (Packard); after 5-10 minutes shaking the plates were read for 1 min in a Packard TOP-Count radioactivity reader.

IC₅₀ Determination: I

Inhibitors were tested at different concentrations ranging from 0.0005 to 10 μM. Experimental data were analyzed by the computer program Assay Explorer using the four parameter logistic equation:
y=bottom+(top−bottom)/(1+10ˆ((log IC₅₀−x)*slope))
where x is the logarithm of the inhibitor concentration, y is the response; y starts at bottom and goes to top with a sigmoid shape.
In addition the selected compounds have been characterized for specificity on Cdk2A, on a panel of ser/threo kinases strictly related to cell cycle (Cdk2/cyclin E, Cdk1/cyclin B1, Cdk4/Cyclin D1, Cdk5/p25), on IGF1-R, Aurora-2, AKT1.
   Inhibition Assay of Cdk2/Cyclin A Activity
   Kinase Reaction:

1.5 μM histone H1 substrate, 25 μM ATP (0.2 μCi P33y-ATP), 30 ng of baculovirus co-expressed Cdk2/Cyclin A, 10 μM inhibitor in a final volume of 100 μl buffer (TRIS HCl 10 mM pH 7.5, MgCl₂ 10 mM, 7.5 mM DTT) were added to each well of a 96 U bottom well plate. After 10 min at 37° C. incubation, reaction was stopped by 20 μl EDTA 120 mM.

Capture:

100 μl were transferred from each well from the kinase reaction to MultiScreen plate, to allow substrate binding to phosphocellulose filter. Plates were then washed 3 times with 150 μl/well PBS Ca⁺⁺/Mg⁺⁺ free and filtered by MultiScreen filtration system.

Detection:

Filters were allowed to dry at 37° C., then 100 μl/well scintillant were added and 33P labeled histone H1 was detected by radioactivity counting in the Top-Count instrument. Results: data were analyzed and expressed as % inhibition referred to total activity of enzyme (=100%).

All compounds showing inhibition ≧50% were further analyzed in order to study and define potency (IC₅₀) as well as the kinetic-profile of inhibitor through Ki calculation. IC₅₀ determination: the protocol used was the same described above, where inhibitors were tested at different concentrations ranging from 0.0045 to 10 μM. Experimental data were analyzed by the computer program GraphPad Prizm using the four parameter logistic equation:
y=bottom+(top−bottom)/(1+10ˆ((log IC₅₀−x)*slope))
where x is the logarithm of the inhibitor concentration, y is the response; y starts at bottom and goes to top with a sigmoid shape.
Ki Calculation:

Either the concentration of ATP and histone H1 substrate were varied: 4, 8, 12, 24, 48 μm for ATP (containing proportionally diluted P³³ μ-ATP) and 0.4, 0.8, 1.2, 2.4, 4.8 μM for histone were used in absence and presence of two different, properly chosen inhibitor concentrations.

Experimental data were analyzed by the computer program “SigmaPlot” for Ki determination, using a random bireactant system equation:

$$v = \frac{V max \frac{( A ) ( B )}{a K A K B}}{1 + \frac{( A )}{K A} + \frac{( B )}{K B} + \frac{( A ) ( B )}{a K A K B}}$$

where A=ATP and B=histone H1.
   Inhibition Assay of Cdk2/Cyclin E Activity
   Kinase Reaction:

1.5 μM histone H1 (Sigma #H-5505) substrate, 25 μM ATP (0.2 μCi P³³γ-ATP), 15 ng of baculovirus co-expressed cdk2/GST-Cyclin E, suitable concentrations of inhibitor in a final volume of 100 μl buffer (TRIS HCl 10 mM pH 7.5, MgCl₂ 10 mM, 7.5 mM DTT+0.2 mg/ml BSA) were added to each well of a 96 U bottom well plate. After 10 min at 37° C. incubation, reaction was stopped by 20 μl EDTA 120 mM.

Capture:

100 μl were transferred from each well to MultiScreen plate, to allow substrate binding to phosphocellulose filter. Plates were then washed 3 times with 150 μl/well PBS Ca⁺⁺/Mg⁺⁺ free and filtered by MultiScreen filtration system.

Detection:

Filters were allowed to dry at 37° C., then 100 μl/well scintillant were added and ³³P labeled histone H1 was detected by radioactivity counting in the Top-Count instrument.

Inhibition Assay of Cdk1/Cyclin B1 Activity

Kinase reaction: 1.5 μM histone H1 (Sigma #H-5505) substrate, 25μμ ATP (0.2 μCi P³³μ-ATP), 30 ng of baculovirus co-expressed Cdk1/Cyclin B1, suitable concentrations of inhibitor in a final volume of 100 μl buffer (TRIS HCl 10 mM pH 7.5, MgCl₂ 10 mM, 7.5-mM DTT+0.2 mg/ml BSA) were added to each well of a 96 U bottom well plate. After 10 min at 37° C. incubation, reaction was stopped by 20 μl EDTA 120 mM.

Capture:

100 μl were transferred from each well to MultiScreen plate, to allow substrate binding to phosphocellulose filter. Plates were then washed 3 times with 150 μl/well PBS Ca⁺⁺/Mg⁺⁺ free and filtered by MultiScreen filtration system.

Detection:

Filters were allowed to dry at 37° C., then 100 μl/well scintillant were added and ³³P labeled histone H1 was detected by radioactivity counting in the Top-Count instrument.

Inhibition Assay Cdk4/Cyclin D1 Activity

Kinase Reaction:

0.4 μM mouse GST-Rb (769-921) (#sc-4112 from Santa Cruz) substrate, 10 μM ATP (0.5 μCi P³³μ-ATP), 100 ng of baculovirus expressed GST-Cdk4/GST-Cyclin D1, suitable concentrations of inhibitor in a final volume of 50 μl buffer (TRIS HCl 10 mM pH 7.5, MgCl₂ 10 mM, 7.5 mM DTT+0.2 mg/ml BSA) were added to each well of a 96 U bottom well plate. After 40 min at 37° C. incubation, reaction was stopped by 20 μl EDTA 120 mM.

Capture:

60 μl were transferred from each well to MultiScreen plate, to allow substrate binding to phosphocellulose filter. Plates were then washed 3 times with 150 ul/well PBS Ca⁺⁺/Mg⁺⁺ free and filtered by MultiScreen filtration system.

Detection:

Filters were allowed to dry at 37° C., then 100 μl/well scintillant were added and ³³P labeled Rb fragment was detected by radioactivity counting in the Top-Count instrument.

Inhibition Assay of Cdk5/p25 Activity

The inhibition assay of Cdk5/p25 activity was performed according to the following protocol:

Kinase Reaction:

1.0 μM biotinylated histone peptide substrate, 0.25 μCi P33g-ATP, 4 nM Cdk5/p25 complex, 0-100 μM inhibitor in a final volume of 100 μl buffer (Hepes 20 mM pH 7.5, MgCl₂ 15 mM, 1 mM DTT) were added to each well of a 96 U bottom well plate. After 20 min at 37° C. incubation, the reaction was stopped by the addition of 500 μg SPA beads in phosphate-buffered saline containing 0.1% Triton X-100, 50 μM ATP and 5 mM EDTA. The beads were allowed to settle, and the radioactivity incorporated in the 33P-labelled peptide was detected in a Top Count scintillation counter.

Results:

Data were analyzed and expressed as % Inhibition using the formula:
100×(1−(Unknown−Bkgd)/(Enz. Control−Bkgd))
IC₅₀ values were calculated using a variation of the four parameter logistics equation:
   Y=100[1+10 ˆ((Log EC50−X)*Slope)]
Where X=log(μM) and Y=% Inhibition.
   Inhibition Assay of IGF1-R Activity

The inhibition assay of IGF1-R activity was performed according to the following protocol:

Kinase Reaction:

10 μM biotinylated MBP (Sigma cat. #M-1891) substrate, 0-20 μM inhibitor, 6 μM cold ATP, 2 nM ³³P-ATP, and 22.5 ng IGF1-R (pre-incubated for 30 min at room temperature with cold 60 μM cold ATP) in a final volume of 30 μl buffer (50 mM HEPES pH 7.9, 3 mM MnCl₂, 1 mM DTT, 3 μM NaVO₃) were added to each well of a 96 U bottom well plate. After incubation for 35 min at room temperature, the reaction was stopped by addition of 100 μl PBS buffer containing 32 mM EDTA, 500 μM cold ATP, 0.1% Triton X100 and 10 mg/ml streptavidin coated SPA beads. After 15 min incubation, 110 μl of suspension were withdrawn and transferred into 96-well OPTIPLATEs containing 100 μl of 5M CsCl. After 4 hours, the plates were read for 2 min in a Packard TOP-Count radioactivity reader.

Results: Experimental data were analyzed with the program GraphPad Prizm.

Inhibition Assay of Aurora-2 Activity

The inhibiting activity and the potency of selected compounds was determined through a method of assay based on the use of the streptavidin scintillation proximity assay beads (amershampharmacia biotech) run in a 96 well plates. At the end of the reaction, the biotinylated peptide substrate was captured with the beads and subsequently allowed to stratify using CsCl₂. When a radioactivity labeled phosphate moiety was transferred by the kinase to the beads-bound peptide, light emitted was measured in a scintillation counter. The inhibition assay of Aurora-2 activity was performed in 96 wells plate according to the following protocol:

Kinase Reaction:

8μμ biotinylated peptide (4 repeats of LRRWSLG), 10μμ ATP (0.5 μCi P³³g-ATP), 10 nM Aurora2, 10μμ inhibitor in a final volume of 60 μl buffer (HEPES 50 mM pH 7.0, MgCl₂ 10 mM, 1 mM DTT, 0.125 mg/ml BSA, 3 μM orthovanadate) were added to each well of a 96 U bottom well plate. After 30 minutes at room temperature incubation, reaction was stopped and biotinylated peptide captured by adding 100 μl of bead suspension.

Stratification:

100 μl of CsCl₂ 7.5 M were added to each well and let stand one hour before radioactivity was counted in the Top-Count instrument.

Results: data were analyzed and expressed as % inhibition referred to total activity of enzyme (=100%).

All compounds showing inhibition ≧60% were further analyzed in order to study the potency of the inhibitor through IC₅₀ calculation. The protocol used was the same described above, except that serial dilution of the inhibitor was used. Experimental data were fitted by nonlinear regression using the following equation:

$$v = {v}_{0} + \frac{( {v}_{0} - {v}_{b} )}{1 + {10}^{n ( log / {C}_{50} - log [ l ] )}}$$

with v_{b} as the baseline velocity, v as the observed reaction velocity, vₒ as the velocity in the absence of inhibitors, and [I] as the inhibitor concentration.
   Inhibition Assay of AKT-1 Activity

Test compounds are prepared as a 10 mM solution in 100% DMSO and distributed into 96 well plates:
i—for % inhibition studies, individual dilution plates at 1 mM, 100 μM and 10 μM are prepared in 100% DMSO, then diluted at a 3× concentration (30, 3 and 0.3 μM) in ddH₂O, 3% DMSO. A Multimek 96 (Beckman) is used for compound pipetting into test plates
ii—for IC₅₀ determination, compounds are diluted to 1 mM in 100% DMSO and plated into the first column of a microtiter plate (A1 to G1), 100 μl. Well H1 is left empty for the internal standard.
A Biomek 2000 (Beckman) is used for serial 1:3 dilutions in water, 3% DMSO, from column A1 to A10 and for all the 7 compounds in the plate. In a standard experiment, the highest concentration of all compounds is 30 μM that is diluted in the final test mixture at 10 μM.

Columns 11 and 12 are left available for total activity reference and background evaluation.

Assay Scheme:

U bottom test plates are prepared either with 10 μl of the compound dilution (3×) per well, or 3% DMSO/water, and then placed onto a PlateTrak robotized station (Packard) together with one reservoir for the Enzyme mix (3×) and one for the ATP mix (3×). As the test starts, the robot (PlateTrak system, Perkin Elmer) takes 10 μl of ATP mix, makes an air gap inside the tips (10 μl) and aspirates 10 μl of Enzyme mix. The following dispensation into the plates allows the kinase reaction to start upon 3 cycles of mixing done by the robot itself.

At this point, the correct concentration is restored for all reagents. The robot incubates the plates for 60 minutes at room temperature, and then stops the reaction by pipetting 150 μl of Dowex resin into the reaction mix. It is essential to keep the resin well stirred before addition to the plates.

The resin is left another 60 minutes to settle down; the robot then takes 50 μl of supernatant from each well and dispenses them into an Optiplate (Packard) with 150 μl of Microscint 40 (Packard).

Counting:

Optiplates, covered by a plastic film to avoid radioactive spilling, are then mixed 10 minutes before counting in a Packard Top Count.

The compounds of the present invention can be administered either as single agents or, alternatively, in combination with known anticancer treatments such as radiation therapy or chemotherapy regimen in combination with cytostatic or cytotoxic agents, antibiotic-type agents, alkylating agents, antimetabolite agents, hormonal agents, immunological agents, interferon-type agents, cyclooxygenase inhibitors (e.g. COX-2 inhibitors),. matrixmetalloprotease inhibitors, telomerase inhibitors, tyrosine kinase inhibitors, anti-growth factor receptor agents, anti-HER agents, anti-EGFR agents, anti-angiogenesis agents (e.g. angiogenesis inhibitors), farnesyl transferase inhibitors, ras-raf signal transduction pathway inhibitors, cell cycle inhibitors, other cdks inhibitors, tubulin binding agents, topoisomerase I inhibitors, topoisomerase II inhibitors, and the like.

If formulated as a fixed dose, such combination products employ the compounds of this invention within the dosage range described below and the other pharmaceutically active agent within the approved dosage range.

Compounds of formula (I) can be used sequentially with known anticancer agents when a combination formulation is inappropriate.

The compounds of formula (I) of the present invention, suitable for administration to a mammal, e.g., to humans, can be administered by the usual routes and the dosage level depends upon the age, weight, conditions of the patient and administration route. For example, a suitable dosage adopted for oral administration of a compound of formula (I) can range from about 10 to about 500 mg per dose, from 1 to 5 times daily. The compounds of the invention can be administered in a variety of dosage forms, e.g., orally, in the form tablets, capsules, sugar or film coated tablets, liquid solutions or suspensions; rectally in the form suppositories; parenterally, e.g., intramuscularly, or through intravenous and/or intrathecal and/or intraspinal injection or infusion.

Another aspect of the invention relates to pharmaceutical compositions comprising a compound of formula (I) or a pharmaceutically acceptable salt thereof in association with a pharmaceutically acceptable carrier. The pharmaceutically acceptable carrier can be an excipeint such as a diluent. The pharmaceutical compositions containing the compounds of the invention are usually prepared following conventional methods and are administered in a suitable pharmaceutical form.

For example, the solid oral forms can contain, together with the active compound, diluents, e.g., lactose, dextrose saccharose, sucrose, cellulose, corn starch or potato starch; lubricants, e.g., silica, talc, stearic acid, magnesium or calcium stearate, and/or polyethylene glycols; binding agents, e.g., starches, arabic gum, gelatine methylcellulose, carboxymethylcellulose or polyvinyl pyrrolidone; disintegrating agents, e.g., starch, alginic acid, alginates or sodium starch glycolate; effervescing mixtures; dyestuffs; sweeteners; wetting agents such as lecithin, polysorbates, laurylsulphates; and, in general, non-toxic and pharmacologically inactive substances used in pharmaceutical formulations. These pharmaceutical preparations can be manufactured in known manner, for example, by means of mixing, granulating, tabletting, sugar-coating, or film-coating processes. The liquid dispersions for oral administration can be syrups, emulsions or suspensions.

As an example, the syrups can contain, as carrier, saccharose or saccharose with glycerine and/or mannitol and sorbitol. The suspensions and the emulsions can contain, as examples of carriers, natural gum, agar, sodium alginate, pectin, methylcellulose, carboxymethylcellulose, or polyvinyl alcohol.

The suspension or solutions for intramuscular injections can contain, together with the active compound, a pharmaceutically acceptable carrier, e.g., sterile water, olive oil, ethyl oleate, glycols, e.g., propylene glycol and, if desired, a suitable amount of lidocaine hydrochloride.

The solutions for intravenous injections or infusions can contain, as a carrier, sterile water. Preferably, they can be in the form of sterile, aqueous, isotonic, saline solutions or they can contain propylene glycol as a carrier.

The suppositories can contain, together with the active compound, a pharmaceutically acceptable carrier, e.g., cocoa butter, polyethylene glycol, a polyoxyethylene sorbitan fatty acid ester surfactant or lecithin.

With the aim to better illustrate the present invention, without posing any limitation to it, the following examples are now given.

General Methods

Flash Chromatography was performed on silica gel (Merck grade 9395, 60A). HPLC was performed on Waters X Terra RP 18 (4.6×50 mm, 3.5 μm) column using a Waters 2790 HPLC system equipped with a 996 Waters PDA detector and Micromass mod. ZQ single quadrupole mass spectrometer, equipped with an electrospray (ESI) ion source. Mobile phase A was ammonium acetate 5 mM buffer (pH 5.5 with acetic acid/acetonitrile 95:5), and Mobile phase B was H₂O/acetonitrile (5:95). Gradient from 10 to 90% B in 8 minutes, hold 90% B 2 minutes. UV detection at 220 nm and 254 nm. Flow rate 1 ml/min. Injection volume 10 μl. Full scan, mass range from 100 to 800 amu. Capillary voltage was 2.5 KV; source temp. was 120° C.; cone was 10 V. Retention times (HPLC r.t.) are given in minutes at 220 nm or at 254 nm. Mass are given as m/z ratio.

When necessary, compounds have been purified by preparative HPLC on a Waters Symmetry C18 (19×50 mm, 5 μm) column using a Waters preparative HPLC 600 equipped with a 996 Waters PDA detector and a Micromass mod. ZMD single quadrupole mass spectrometer, electron spray ionization, positive mode. Mobile phase A was water 0.01 % TFA, and Mobile phase B was acetonitrile. Gradient from 10 to 90% B in 8 min, hold 90% B 2 min. Flow rate 20 ml/min.

1H-NMR spectrometry was performed on a Mercury VX 400 operating at 400.45 MHz equipped with a 5 mm double resonance probe [1H (15N-31P) ID_PFG Varian].

The compounds of formula (I), having an asymmetric carbon atom and obtained as racemic mixture, were resolved by HPLC separation on chiral columns. In particular, for example, preparative columns CHIRALPACK® AD, CHIRALPACK® AS, CHIRALCELL® OJ can be used.

### EXAMPLE 1

### Preparation of ethyl 5-[(tert-butoxycarbonyl)amino]-3-oxopentanoate

1.26 g (6.6 mmol) of N-Boc-β-alanine were dissolved with Meldrum's acid (1 g, 6.9 mmol) and 4-dimethylaminopyridine (1.28 g, 10.5 mmol) in 70 mL of dichloromethane (DCM). The reaction mixture was cooled to 0° C. and a solution of 1.58 g (7.6 mmol) of N,N′-dicyclohexylcarbodiimide in 50 mL of DCM was added dropwise. The mixture was left at 0° C. overnight, during which time tiny crystals of dicyclohexylurea precipitated. After filtration, the reaction mixture was washed 3 times with an aqueous solution of 5% sodium bisulfate and one more time with brine. Organic extracts were dried over sodium sulfate and the solvent was evaporated under vacuum and then dried. The solid was dissolved in ethanol and heated at 70° C. for 6 hours. The solvent was removed and the raw product was purified by flash chromatography over silica gel thus obtaining 650 mg of the title compound as a yellow oil.

H¹NMR (400 MHz, CDCl₃); δ ppm 1.27 (t, 3 H), 1.4 (s, 9 H), 2.78 (t, 2 H), 3.36 (m, 2 H), 3.44 (s, 2 H), 4.2 (q, 2 H), 5.0 (br s, 1 H).

### EXAMPLE 2

### Preparation of ethyl 2-{2-[(tert-butoxycarbonyl)amino]ethyl}-5-pyridin-4-yl-1H-pyrrole-3-carboxylate

540 mg of ethyl 5-[(tert-butoxycarbonyl)amino]-3-oxopentanoate (2.08 mmol) and 208 mg of sodium hydride (60% dispersion oil, 5.2 mmol) dissolved in 20 mL of THF were stirred 1 hour at room temperature and then cooled to 0° C. A suspension of 735 mg (3.67 mmol) of 2-bromo-1-pyridin-4-ylethanone in 10 mL of THF was added dropwise and the mixture was stirred at 0° C. for 4 hours. The resulting solution was dried and then dissolved in 30 mL of ethanol; 500 mg (8.47 mmol) of ammonium acetate were added. The solution was left stirring 5 hours and then dried. The raw product was dissolved in ethyl acetate, washed three times with brine and dried over sodium sulfate. The solvent was removed and the raw product was purified by flash chromatography over silica gel, thus obtaining 280 mg (0.78 mmol, 37%) of the title compound.

1H NMR (400 MHz, DMSO-D6) δppm 1.3 (t, J=7.0 Hz, 4 H) 1.3 (s, 9 H) 3.0 (t, J=7.2 Hz, 2 H) 3.2 (m, 2 H) 4.2 (q, J=7.0 Hz, 2 H) 7.1 (d, J=2.6 Hz, 1 H) 7.6 (d, J=6.4 Hz, 2 H) 8.5 (d, J=6.2 Hz, 2 H) 11.9 (s, 1 H).

HPLC retention time (RT): 4.9 min; ESI (+) MS: m/z 360 (MH+).

### EXAMPLE 3

### Preparation of 4-[5-(2-ammonioethyl)-4-(ethoxycarbonyl)-1H-pyrrol-2-yl]pyridinium dichloride

To a solution of 20 mg of ethyl 2-{2-[(tert-butoxycarbonyl)amino]ethyl}-5-pyridin-4-yl-1H-pyrrole-3-carboxylate, 2 mL of HCl 4 M in dioxane were added. The solution was left shaking 3 hours and then the product was dried under vacuum thus affording the title compound.

1H NMR (400 MHz, DMSO-D6) μ ppm 1.3 (t, J=7.1 Hz, 3 H) 3.2 (m, 4 H) 4.3 (q, J=7.1 Hz, 2 H) 7.6 (d, J=2.6 Hz, 1 H) 8.1 (m, 3 H) 8.3 (d, J=6.3 Hz, 2 H) 8.7 (d, J=5.7 Hz, 2 H) 13.0 (s, 1 H); HPLC RT 2.3 min; ESI (+) MS: m/z 260 (MH+).

### EXAMPLE 4

### Preparation of 2-pyridin-4-yl-1,5,6,7-tetrahydro-4H-pyrrolo[3,2-c]pyridin-4-one hydrochloride

To a solution of 20 mg of 2-[3-(ethoxycarbonyl)-5-pyridin-4-yl-1H-pyrrol-2-yl]ethanaminium chloride in 2 mL of ethanol, about 10 mg of potassium carbonate were added and the solution was refluxed for 16 hours. The mixture was cooled to room temperature, the solvent removed under evaporation and the raw product was purified by flash chromatography over silica gel, thus affording the title compound as a free base. Sometimes, when required, the free base was dissolved in ethanol, treated with 4 N hydrochloric acid in dioxane and diluted with ethyl acetate until precipitation of the hydrochloride salt that was filtered, thus affording the title compound.

¹H NMR (DMSO-d₆/400 MHz) μ ppm 2.94 (t, 2 H, J=6.83 Hz), 3.45 (t, 2 H, J=6.83 Hz), 7.30 (bs, 1 H), 7.59 (s, 1 H), 8.23 (d, 2 H, J=7.08 Hz), 8.71 (d, 2 H, J=7.08 Hz), 12.89 (bs, 1 H).

### EXAMPLE 5

### Preparation of 2,4-dioxo-piperidine-1-carboxylic acid tert-butyl ester

Boc-μ-alanine (25 g, 132 mmol), Meldrum's Acid (1.1 eq., 145 mmol, 20.9 g) and 4-dimethylaminopyridine (DMAP, 1.5 eq., 198 mmol, 24.2 g) were dissolved in 700 mL of dry DCM at 0° C. under nitrogen atmosphere. EDCl hydrochloride (1.2 eq, 158 mmol, 30.4 g) was added. The resulting solution was allowed to reach room temperature and stirred overnight. The reaction mixture was washed (0.5 L×4) with 5% KHSO₄ aqueous solution. Organic layer was dried (Na₂SO₄), filtered and evaporated under vacuum, affording crude [3-(2,2-dimethyl-4,6-dioxo-[1,3]dioxan-5-yl)-3-oxo-propyl]-carbamic acid tert-butyl ester that was dissolved in 600 mL of EtOAc and refluxed for 4 hours. Solvent was reduced to 150 mL under vacuum and the resulting solution was allowed to crystallize at 4° C. overnight. The solid was filtered off and washed with cooled EtOAc affording 18.4 g of 2,4-dioxo-piperidine-1-carboxylic acid tert-butyl ester 86.3 mmol, 65.4% yield.

1H NMR (400 MHz, DMSO-D6) μ ppm 1.44 (s, 9 H) 2.44 (m, 2 H) 3.71 (m, 2 H) 4.95 (s, 1 H) 11.2 (bs, 1 H).

### EXAMPLE 6

### Preparation of piperidine-2,4-dione

A solution of μ-alanine ethylester hydrochloride (13.8 g, 90mmol) in dichloromethane (90 mL) and TEA (13.8 mL, 99 mmol) was stirred at RT for one hour. More TEA (13.8 mL, 99 mmol) was added, the solution was cooled to 0° C. under stirring and ethylmalonylchloride (12.6 mL, 99 mmol) was added dropwise. After one hour at 0° C., the reaction mixture was stirred one hour at RT. A 15% aqueous solution of K₂CO₃ (90 mL) was added and the layers were separated. The organic phase was washed with 10% HCl (90 mL), dried over Na₂SO₄, filtered and concentrated under reduced pressure. The crude material was chromatographed on flash silica gel (450 g, eluant: ethyl acetate/n-hexane 2:1) to give N-(2-ethoxycarbonyl-ethyl)-malonamic acid ethyl ester as a yellow oil (15 g, 64.9 mmol, 72% yield). TLC: DCM/MeOH 20:1, iodine vapours. Sodium metal (610 mg, 26.6 mmol) was dissolved in dry MeOH (25 mL) at RT under stirring and inert atmosphere. After complete dissolution the mixture was stirred 10′ longer, then N-(2-ethoxycarbonyl-ethyl)-malonamic acid ethyl ester (6.15 g, 26.6 mmol) in dry toluene (150 mL) is added dropwise. After addition the reaction mixture was stirred at 90° C. for 6 hours, cooled to RT, water (30 mL) was added and the layers were separated. The organic phase was washed with water (2×10 mL), the joined aqueous phases were acidified with 37% HCl and extracted thoroughly (×20 or more) with a mixture of DCM/MeOH (5:1). After drying over Na₂SO₄ and concentration, 3-methoxycarbonylpiperidin-2,4-dione as a pink solid was obtained (4 g, 23.4 mmol, 88% yield). 3-Methoxycarbonylpiperidin-2,4-dione (4 g, 23.4 mmol) is dissolved in acetonitrile containing 1% of water (250 mL) and refluxed 4 hours. The reaction mixture is concentrated to give piperidine-2,4-dione, as a yellow solid (2.4 g, 21.2 mmol, 90% yield).

1H NMR (400 MHz, DMSO-D6) δ ppm 2.43-2.49 (m, 2 H) 3.24 (s, 2 H) 3.28-3.45 (m, 2 H) 8.07 (s, 1 H).

### EXAMPLE 7

### Preparation of 6,6-dimethyl-2,4-dioxopiperidine

A solution of ethyl 3-methylbut-2-enoate (1 g, 7.8 mmol) in anhydrous ethanol (12 mL) was cooled to −20° C. and saturated with gaseous ammonia. The tube was sealed and kept at 90° C. for 24 hours. The reaction was cooled to room temperature, bubbled with nitrogen to eliminate the residual ammonia and treated with a 4 N solution of HCl in dioxane (1.9 mL). After 30 minute stirring, the mixture was evaporated under reduced pressure to give ethyl 3-amino-3-methylbutanoate hydrochloride as a grey solid (1.19 g, Y=84%).

¹H NMR (CDCl₃/400 MHz) δ ppm 1.2 (t, 3 H), 1.26 (s, 6 H), 2.65 (s, 2 H), 4.1 (q, 2 H), 8.27 (bs, 3 H).

Ethyl 3-amino-3-methylbutanoate hydrochloride (0.87 g, 4.79 mmol) was suspended on methylene chloride (12 mL) and triethylamine (1.4 mL, 2.1 eq.). The mixture was cooled to 0° C. and treated dropwise with ethyl 3-chloro-3-oxopropanoate (0.64 mL, 1.05 eq.). The reaction was kept at room temperature for 2 hours, diluted with methylene chloride, washed with 1 N HCl and then with 5% NaHCO₃, dried over Na₂SO₄ and evaporated to dryness to obtain ethyl 3-[(3-ethoxy-3-oxopropanoyl)amino]-3-methylbutanoate (1.2 g, Y=97%) as a red oil.

¹H NMR (DMSO-d₆/400 MHz) δ ppm 1.11-1.21 (m, 6 H), 1.29 (s, 6 H), 2.71 (s, 2 H), 3.14 (s, 2 H), 3.95-4.15 (m, 4 H), 7.75 (bs, 1 H).

To a solution of sodium ethoxide, obtained from sodium metal (0.122 g, 5.55 mmol) in anhydrous ethanol (7 mL), a solution of ethyl 3-[(3-ethoxy-3-oxopropanoyl)amino]-3-methylbutanoate (1.2 g, 4.62 mmol) in dry toluene (7 mL) was added dropwise at room temperature, under stirring. The reaction mixture was heated at 80° C. for 2 hours then it was concentrated to reduced volume and the residue was dissolved in toluene (15 mL). The organic phase was extracted with water (40 mL), the aqueous phase was acidified to pH 2-3 with 1 N HCl and extracted with ethyl acetate (4×50 mL). The organic phase was washed with brine, dried over anhydrous sodium sulphate and concentrated to give ethyl 6,6-dimethyl-2,4-dioxopiperidine-3-carboxylate as a yellow solid (0.7 g, Y=71%) which was used for the next step without further purification. Ethyl 6,6-dimethyl-2,4-dioxopiperidine-3-carboxylate (0.69 g, 3.23 mmol) was dissolved in acetonitrile containing 1% of water (15 mL) and the resulting solution was refluxed for 2 hours. After evaporating to dryness, the crude material was suspended in isopropyl ether, kept under vigorous stirring and filtered to give the title compound (387 mg, Y=85%) as a light brown solid.

¹H NMR (DMSO-d₆/400 MHz) μ ppm 1.18 (s, 6 H), 2.49 (bs, 2 H), 3.13 (bs, 2 H), 8.13 (bs, 1 H).

### EXAMPLE 8

### Preparation of 5-isopropylpiperidine-2,4-dione

Ethyl 2-cyano-3-methylbut-2-enoate (5.0 g, 32.6 mmol) was dissolved in 320 mL of absolute EtOH. 700 mg of PtO₂ and 12 mL of 4M HCl were added. The reaction mixture was hydrogenated at room temperature for 5 hours (30 psi). Filtration on a celite pad and evaporation of the solvent afforded crude ethyl 2-(aminomethyl)-3-methylbutanoate hydrochloride (quantitative yield).

1H NMR (400 MHz, DMSO-D6) δ ppm 0.90 (d, J=6.83 Hz, 3 H) 0.93 (d, J=6.83 Hz, 3 H) 1.24 (t, J=7.13 Hz, 3 H) 1.92-2.06 (m, 1 H) 2.53-2.60 (m, 1 H) 2.84-3.17 (m, 2 H) 4.05-4.24 (m, 2 H) 7.84 (s, 3 H); ESI (+) MS: m/z 160 (MH+).

Crude ethyl 2-(aminomethyl)-3-methylbutanoate hydrochloride was dissolved in 200 mL of dry DCM and DIPEA was added (14 mL, 2.5 eq). After cooling to 0° C., ethyl 3-chloro-3-oxopropanoate was added (6.3 mL, 35.4 mmol). After stirring at room temperature overnight, the reaction mixture was diluted with DCM and washed with aq. KHSO₄ 5% (×2), aq. NaHCO₃ sat. sol. (×2) and brine. The organic layer was dried over Na₂SO₄, filtered and evaporated to dryness. Column chromatography (hexane/EtOAc=7/3→1/1) afforded 8.35 g (30.55 mmol, 93.4% yield) of ethyl 2-{[(3-ethoxy-3-oxopropanoyl)amino]methyl}-3-methylbutanoate.

1H NMR (400 MHz, DMSO-D6) δ ppm 0.89 (d, J=6.82 Hz, 3 H) 0.93 (d, J=6.83 Hz, 3 H) 1.20 (m, 6 H) 1.82-1.90 (m, 1 H) 2.35 (m, 1 H) 3.19-3.33 (2 m, 4 H) 4.06 (m, 4 H) 8.11 (t, J=5.12 Hz, 1 H); ESI (+) MS: m/z 274 (MH+).

Crude ethyl 2-{[(3-ethoxy-3-oxopropanoyl)amino]methyl}-3-methylbutanoate (8.35 g, 30.55 mmol) was dissolved in 215 mL of dry toluene and heated to 100° C. 6.9 mL of sodium methoxide 30 wt. % solution in methanol were added (36 mmol) and the reaction mixture was refluxed for 4 hours. After cooling at room temperature, the organic phase was washed with water (×2). The aqueous layers were collected, acidified (10% HCl) and extracted with DCM (×4). The organic layers were collected and evaporated to dryness. The crude was treated with 250 mL of 10% AcOH in water and refluxed for 3 hours. The reaction mixture was neutralized with NaHCO₃ (˜ pH 7) and extracted with DCM (×5). The organic layers were collected, dried (Na₂SO₄), filtered and evaporated to dryness. Column chromatography (DCM/EtOH=97/3) afforded 2.35 g of target product (15.14 mmol, 49.6% yield).

1H NMR (400 MHz, DMSO-D6) δ ppm 0.85 (d, J=6.83 Hz, 3 H) 0.94 (d, J=6.95 Hz, 3 H) 2.07-2.17 (m, 1 H) 2.25-2.33 (m, 1 H) 3.09-3.41 (m, 4 H) 8.03 (s, 1 H).

ESI (+) MS: m/z 156 (MH+).

By working in an analogous way as in Example 8 and starting from 2-cyanopropionic acid ethyl ester the following compound in Example 9 was also obtained:

### EXAMPLE 9

### 5-Methyl-2,4-dioxo-piperidine-1-carboxylic acid tert-butyl ester

1H NMR (400 MHz, DMSO-D6) μ ppm 1.09 (d, J=6.95 Hz, 3 H) 1.45 (s, 9 H) 2.49-2.59 (m, 1 H) 3.49-3.57 (m, 1 H) 3.68-3.77 (m, 1 H) 4.92 (s, 1 H) 11.17 (s, 1 H).

ESI (+) MS: m/z 128 (MH+).

### EXAMPLE 10

### 6Benzylpiperidine-2,4-dione

A mixture of beta-homophenylalanine (9.1 g, 50.94 mmol), di-tert-butyl dicarbonate (12.2 g, 56.1 mmol), dioxane (180 mL), water (18 mL) and triethylamine (8.5 mL) was stirred at RT overnight. After concentration and multiple strippings with toluene, 3-[(tert-butoxycarbonyl)amino]4-phenylbutanoic acid was obtained as an oil and used directly in the next step. It was dissolved in dry dichloromethane (370 mL), Meldrum acid (8.1 g, 56.1 mmol) and DMAP (9.7 g, 79 mmol) were added to it, the mixture was cooled to −5° C. and dicyclohexylcarbodiimide (12.6 g, 61 mmol) was added. After addition the reaction mixture was kept in refrigerator overnight. The precipitate was filtered off and washed with dichloromethane. The filtrate was diluted with ethylacetate, washed in sequence with 10% aq KHSO₄, water, brine then concentrated to yield crude tert-butyl 1-benzyl-3-(2,2-dimethyl-4,6-dioxo-1,3-dioxan-5-yl)-3-oxopropylcarbamate that was dissolved in ethylacetate (250 mL) and refluxed 2 h. After concentration and treatment with diisopropylether the crystallized compound was filtered and washed to give tert-butyl 2-benzyl4,6-dioxopiperidine-1-carboxylate as a white powder in 75% overall yield.

The t-butoxycarbonyl group could be removed by acidic treatment (4M HCl in dioxane) at RT.

1H NMR (400 MHz, DMSO-D6) δ ppm 2.32 (dd, J=15.73, 8.17 Hz, 1 H) 2.42 (dd, J=16.34, 4.76 Hz, 1 H) 2.66-2.74 (m, 1 H) 2.87-3.02 (m, 2 H) 3.25-3.40 (m, 1 H) 3.84-3.93 (m, 1 H) 7.20-7.36 (m, 5 H) 8.14 (s, 1 H).

By working in an analogous way as in Example 10, the following compounds in Examples 11-12 were also obtained:

### EXAMPLE 11

### 5,5-dimethylpiperidine-2,4-dione

1H NMR (400 MHz, DMSO-D6) δ ppm 1.0 (s, 6 H) 3.15 (s, 2 H) 3.25 (s, 2 H) 8.0 (s, 1 H).

### EXAMPLE 12

### 2-Isobutyl-4,6-dioxo-piperidine-1-carboxylic acid tert-butyl ester

1H NMR (400 MHz, DMSO-D6) δ ppm 0.87 (at, J=6.59 Hz, 6 H) 1.30-1.56 (m, 3 H) 1.41 (s, 9 H) 2.13 (d, J=17.58 Hz, 1 H) 2.83 (dd, J=17.73, 6.30 Hz, 1 H) 4.36 (add, J=6.74, 6.15 Hz, 1 H) 4.93 (s, 1 H) 11.06 (bs, 1 H)

### EXAMPLE 13

### Preparation of 5-ethylpiperidine-2,4-dione

To a solution of tert-butyl 2,4-dioxopiperidine-1-carboxylate (1.92 g, 9.0 mmol), in dry THF (65 mL) and cooled to −20° C. under nitrogen, lithium bis(trimethylsilyl)amide (LiHMDS) (27 mL of 1 M solution in THF) was added dropwise. After 20 min stirring, 2.53 mL (4.9 g, 31.3 mmol) of iodoethane were added and the solution was stirred at −20° C. for 2 hours. The reaction mixture was poured in 5% aq KHSO₄ and extracted with DCM (×2). The collected organic layers were washed with water, dried over Na₂SO₄ and evaporated to dryness. The residue was purified by column chromatography (n-hexane/EtOAc 1:1) affording 1.4 g of 5-ethyl-2,4-dioxo-piperidine-1-carboxylic acid tert-butyl ester (5.8 mmol, 64%); ESI (+) MS: m/z 242 (MH+).

The compound was dissolved in TFA (10 mL) and the resulting solution was stirred at room temperature for 2 hours. After evaporation, the residue was purified by column chromatography (n-hexane/EtOAc 1:2) affording 5-ethylpiperidine-2,4-dione (52% yield).

1H NMR (400 MHz, DMSO-D6) δ ppm 0.89 (t, J=7.56 Hz, 3 H), 1.35 (m, 1 H), 1.69 (m, 1 H), 2.39 (m, 1 H), 3.14-3.38 (m, 4 H), 8.05 (s, 1H); ESI (+) MS: m/z 142 (MH+)

### EXAMPLE 14

### Preparation of 2-bromo-1-pyridin-ylethanone hydrobromide

To a stirred solution of 4-acetylpyridine (10 mL, 90 mmols) in glacial acetic acid (40 mL) and 48% hydrobromic acid (15 mL), bromine (4.65 mL, 90 mmols) in glacial acetic acid (10 mL) was added dropwise. After addition, the solution was stirred at room temperature overnight. The white precipitate was filtered off and washed with absolute ethanol, thus obtaining the title compound (22.2 g Y=90%) as a white solid containing traces of dibromoderivative, that was used as such in the next step.

¹H NMR (DMSO-d₆/400 MHz) δ ppm 5.05 (s, 2 H) 8.15 (d, 2 H) 9.0 (d, 2 H).

### EXAMPLE 15

### Preparation of 2-bromo-1-(3-fluoropyridin-4-yl)ethanone hydrobromide

Into a stirred solution of 3-fluoropyridine (14 g, 144.2 mmol) in anhydrous THF (150 mL), cooled to −78° C. and under argon, 79.2 mL (158.6 mmol) of a 2N solution of lithiumdiisopropylamide (LDA) in n-heptane, THF, ethylbenzene, were slowly dropped in about 1 h. After stirring for 2.5 h a cooled solution (ca. 0° C.) of acetaldehyde (8.9 mL, 158.5 mmol) in 25 mL of anhydrous THF was slowly dropped and the reaction mixture was stirred at −78° C. for 1.5 h. The solution was warmed to −30° C. and a solution of ammonium chloride (150 g) in 700 mL of water was added. The mixture was extracted with ethylacetate (3×400 mL) and the organic layers were washed with brine (4×200 mL) and dried over sodium sulfate. After concentration the oil was crystallized with n-hexane (40 mL) and 15.6 g (76% yield) of 1-(3-fluoropyridin-4-yl)ethanol were obtained. A mixture of 1-(3-fluoropyridin-4-yl)ethanol (10 g, 70.3 mmol) and commercial activated MnO₂ (8 g, 92.1 mmol) in toluene (100 mL) were refluxed until disappearance of starting material. After cooling the mixture was filtered on a bed of celite, the cake washed with toluene and the organic phases concentrated to give 3-fluoro-4-acetyl pyridine (6.9 g, 70%) that was used directly in the next step. To a stirred solution of 3-fluoro-4-acetylpyridine (5.3 g, 38.1 mmol) in glacial acetic acid (14 mL) and 48% hydrobromic acid (5.3 mL), bromine (2 mL, 38 mmol) in glacial acetic acid (5.3 mL) was added slowly and dropwise. After addition, the solution was stirred at 60° C. for 2.5 h then it was cooled down and ethylacetate (70 mL) was added. After 30′ stirring the mixture was filtered and the solid was washed thoroughly with ethylacetate and dried. The title compound was obtained in 82% yield (9.4 g).

¹H NMR (DMSO-d₆/400 MHz) δ ppm 4.88 (s, 2 H) 7.83 (dd, 1 H) 8.62 (dd, 1 H) 8.81 (d, 1 H).

### EXAMPLE 16

### 1-(2-aminopyrimidin-4-yl)-2-bromoethanone hydrobromide

A mixture of 3,3-dimethoxy-2-butanone (25 g, 189.16 mmol) and N,N-dimethylformamide dimethylacetal (22.5 g, 189.16 mmol) were stirred at 110° C. for 30 hours and then distilled (115° C., 1 mmHg) thus obtaining 1-(dimethylamino)-4,4-dimethoxypent-1-en-3-one, as a yellow solid (27.3 g, 146 mmol, 77%). Onto a solution of sodium (3.48 g, 151.67 mmol) in anhydrous ethanol (400 mL), solid guanidine hydrochloride (14.5 g, 151.67 mmol) was added at r.t., to give a white suspension into which a solution of 1-(dimethylamino)-4,4-dimethoxypent-1-en-3-one (28.4 g, 151.67 mmol) in anhydrous ethanol (50 mL) was added. The mixture was refluxed for 19 hours. After cooling, the precipitate was filtered and washed with ethanol and with plenty of water, thus obtaining a white solid (8.56 g). The ethanolic solutions were concentrated to dryness, taken up with boiling ethyl acetate (1000 mL), filtered while hot and then cooled to yield a second crop. Total amount of 4-(1,1-dimethoxyethyl)pyrimidin-2-amine: 17.66 g, 63.5%. A solution of the said amine (17.5 g, 95.5 mmol) in formic acid was stirred at r.t. for 6 hours and concentrated to dryness and the residue was stirred in ethanol (50 mL) and then filtered thus obtaining 1-(2-aminopyrimidin-4-yl)ethanone (9.2 g, 70%). To a solution of 1-(2-aminopyrimidin-4-yl)ethanone (412 mg, 3 mmol) in glacial acetic acid (1 mL) and 48% aq. HBr (0.3 mL), bromine (0.153 mL) in acetic acid (0.4 mL) was added and the resulting orange solution was stirred at r.t. for 15 hours. After diluting with ethyl acetate (15 mL) the precipitate was filtered and washed with ethyl acetate thus affording the title compound as a whitish solid (580 mg, 65%).

¹H NMR (DMSO-d₆/400 MHz) δ ppm: 4.9 (s, 2 H), 7.0 (d, 2 H), 8.5 (d, 2 H).

### EXAMPLE 17

### 2-Pyridin-4-yl-1,5,6,7-tetrahydro-pyrrolo[3,2-c]pyridin-4-one (alternative method)

Bromoacetylpyridine hydrobromide (3.3 g, 11.78 mmol), piperidindione (2 g, 17.68 mmol) and ammonium acetate (3.63 g, 47.1 mmol) were dissolved in anhydrous ethanol (54 mL) and stirred at RT overnight. Ethyl acetate (200 mL) was added (precipitate formed) and the mixture was stirred at RT for 30′. The solid was filtered off and discarded, while the solution was concentrated under reduced pressure. The residue (orange-red solid, 4.8 g) was purified by flash chromatography (eluant DCM/MeOH 6:1).

To the obtained pink solid (1.34 g, 6.28 mmol), dissolved in MeOH (140 mL), 4N HCl in dioxane (3.14 mL, 12.56 mmol) was added. The mixture (precipitate) was stirred for 30′, then concentrated under reduced pressure to half of the volume, stirred 30′ and filtered to yield the first crop (1.3 g). The mother liquor was concentrated to 20 mL and the second crop filtered out (0.12 g). The two crops were joined and washed twice with 95% EtOH: first with 35 mL and 2 hours stirring, the second with 25 mL of ethanol. The collected solid was dried to yield 1.21 g of desired compound (41.1% yield, purity>90%).

By working in an analogous way and starting from the corresponding bromoketoheteroaryl the following compounds were also obtained:

### EXAMPLE 18

### 2-(3-fluoropyridin-4-yl)-1,5,6,7-tetrahydro-4H-pyrrolo[3,2-c]pyridin-4-one hydrochloride

1H NMR (400 MHz, DMSO-D6) δ ppm 2.91 (m, 2 H) 3.41 (m, 2 H) 7.08 (bs, 1 H) 7.19 (bs, 1 H) 7.90 (dd, J=5.60 Hz, 7.19 Hz, 1 H) 8.48 (d, J=5.00 Hz, 1 H) 8.70 (d, J=4.15 Hz, 1 H) 12.15 (bs, 1 H).

### EXAMPLE 19

### 3-methyl-2-pyridin-4-yl-1,5,6,7-tetrahydro-4H-pyrrolo[3,2-c]pyridin-4-one hydrochloride

1H NMR (400 MHz, DMSO-D6) δ ppm 2.66 (s, 3 H) 2.90 (t, J=6.83 Hz, 2 H) 3.29-3.47 (m, 2 H) 7.24 (s, 1 H) 8.00 (d, J=6.95 Hz, 2 H) 8.71 (d, J=7.19 Hz, 2 H) 12.39 (s, 1 H).

### EXAMPLE 20

### 2-(2-aminopyrimidin-4-yl)-1,5,6,7-tetrahydro-4H-pyrrolo[3,2-c]pyridin-4-one hydrochloride

¹H NMR (DMSO-d₆/400 MHz) δ ppm 2.91 (t, 2 H, J=6.71 Hz), 3.36 (t, 2 H, J=6.71 Hz), 7.27 (d, 1 H, J=6.70 Hz), 7.29 (bs, 1 H), 7.46 (s, 1 H), 7.86 (br, 2 H), 8.21 (d, 2 H, J=6.70 Hz) By working in an analogous way and starting from the corresponding, optionally protected, piperidindiones, the following compounds were also obtained:

### EXAMPLE 21

### 2-(2-aminopyrimidin-4-yl)-7-methyl-1,5,6,7-tetrahydro-4H-pyrrolo[3,2-c]pyridin-4-one hydrochloride

1H NMR (400 MHz, DMSO-D6) δ ppm 1.29 (d, J=6.58 Hz, 3 H) 3.06-3.24 (m, 2 H) 3.41-3.56 (m, 1 H) 7.30 (s, 1 H) 7.35 (d, J=6.83 Hz, 1 H) 7.51 (s, 1 H) 8.03 (s, 2 H) 8.23 (d, J=6.71 Hz, 1 H) 12.24 (s, 1 H).

### EXAMPLE 22

### 2-(2-aminopyrimidin-4-yl)-7,7-dimethyl-1,5,6,7-tetrahydro-4H-pyrrolo[3,2-c]pyridin-4-one hydrochloride

1 H NMR (400 MHz, DMSO-D6) δ ppm 1.37 (s, 6 H) 3.20 (s, 2 H) 7.39-7.43 (m, 1 H) 7.46 (d, J=6.70 Hz, 1 H) 7.52 (s, 1 H) 8.16 (s, 2 H) 8.28 (d, J=6.70 Hz, 1 H) 12.20 (s, 1 H).

### EXAMPLE 23

### 2-(2-Amino-pyrimidin-4-yl)-7-ethyl-1,5,6,7-tetrahydro-pyrrolo[3,2-c]pyridin-4-one

1H NMR (400 MHz, DMSO-D6) δ ppm 0.94 (t, J=6.95 Hz, 3 H) 1.7 (m, 2 H) 2.9 (m, 1 H) 3.3 (m, 2 H) 7.25 (bs, 1 H) 7.35 (d, J=6.83 Hz, 1 H) 7.5 (s, 1 H) 8.04 (bs, 2 H) 8.22 (d, J=6.83 Hz, 1 H) 12.25 (bs, 1 H).

### EXAMPLE 24

### 2-(2-aminopyrimidin-4-yl)-7-isopropyl-1,5,6,7-tetrahydro-4H-pyrrolo[3,2-c]pyridin-4-one

1H NMR (400 MHz, DMSO-D6) δ ppm 0.91 (t, J=6.95 Hz, 6 H) 1.07 (t, J=6.95 Hz, 1 H), 1.96-2.10 (m, 1 H) 2.66-2.74 (m, 1 H) 3.40-3.54 (m, 1 H) 6.28-6.39 (m, 2 H) 6.95 (d, J=5.37 Hz, 1 H) 6.97 (s, 1 H) 7.03 (d, J=2.19 Hz, 1 H) 8.16 (d, J=5.24 Hz, 1 H) 11.64 (s, 1 H).

The racemate was subjected to chiral separation so to obtain the pure enantiomers. Chiral chromatography was performed on Chiralpack® AS column (5×50 cm). Mobile phase: nHexane/iPropanol/Ethanol/Methanol 30/30/30/10

Analytical Conditions:

Chiralpack® AS column with precolumn, mobile phase nHexane/iPropanol/Ethanol/Methanol 30/30/30/10

### EXAMPLE 25

### (7R and 7S)-2-(2-Amino-pyrimidin-4-yl)-7-isopropyl-1,5,6,7-tetrahydro-pyrrolo[3,2-c]pyridin-4-one

e.e.>99%

### EXAMPLE 26

### 2-(2-aminopyrimidin-4-yl)-6-isobutyl-1,5,6,7-tetrahydro-4H-pyrrolo[3,2-c]pyridin-4-one hydrochloride

1H NMR (400 MHz, DMSO-D6) δ ppm 0.81-0.97 (m, 6 H); 1.27-1.42 (m, 1 H); 1.42-1.58 (m, 1 H); 1.64-1.81 (m, 1 H); 2.70 (dd, J=16.58, 8.41 Hz, 1 H); 3.04 (dd, J=16.58, 5.24 Hz, 1 H); 3.63-3.80 (m, 1 H); 7.29 (s, 1H); 7.31 (d, J=6.71 Hz, 1 H); 7.51 (s, 1 H); 8.08 (s, 2 H); 8.21 (d, J=6.71 Hz, 1 H); 12.35 (s, 1 H).

### EXAMPLE 27

### 2-(2-Amino-pyrimidin-4-yl)-6-benzyl-1,5,6,7-tetrahydro-pyrrolo[3,2-c]pyridin-4-one

1H NMR (400 MHz, DMSO-D6) δ ppm 2.7-3.2 (m, 4 H) 3.9 (m, 1 H) 7.0-7.05 (2s, 2H) 7.25-7.4 (m, 6 H) 8.1 (bs, 2 H) 8.25 (s, 1 H) 11.8 (bs, 1 H).

### EXAMPLE 28

### 6,6-dimethyl-2-(2-aminopyrimidin-4-yl)-1,5,6,7-tetrahydro-4H-pyrrolo[3,2-c]pyridin-4-one hydrochloride

¹H NMR (DMSO-d₆/400 MHz): δ ppm 1.3 (sc, J=2.7, 0.9, 0.5 Hz, 3 H) 1.5 (sc, J=2.7, 0.9, 0.5 Hz, 3 H) 2.5 (sc, J=15.6, 1.5, 0.9, 0.5 Hz, 1 H) 2.6 (sc, J=15.6, 1.5, 0.9, 0.5 Hz, 1 H) 7.5 (sc, J=0.9 Hz, 1 H) 7.7 (sc, J=4.8 Hz, 1 H) 8.2 (sc, J=4.8 Hz, 1 H) 9.2 (sc, 1 H).

### EXAMPLE 29

### 2-(2-Amino-pyrimidin-4-yl)-1-benzyl-1,5,6,7-tetrahydro-pyrrolo[3,2-c]pyridin-4-one

To a solution of 2-(2-Amino-pyrimidin-4-yl)-4-oxo-1,4,6,7-tetrahydro-pyrrolo[3,2-c]pyridine-5-carboxylic acid tert-butyl ester (300 mg, 0.9 mmol) in anhydrous DMF (5 mL) 60% NaH (58 mg) was added and the mixture was stirred for 2 hours at room temperature. Benzylbromide (0.25 mL) was added and the reaction mixture was stirred overnight. After pouring in water and ethyl acetate the organic layer was dried and concentrated. The residue was purified by flash chromatography over silica gel (eluant: DCM/MeOH 10:1) to yield 2-(2-Amino-pyrimidin-4-yl)-1-benzyl-4-oxo-1,4,6,7-tetrahydro-pyrrolo[3,2-c]pyridine-5-carboxylic acid tert-butyl ester (240 mg, 0.58 mmol) that was subdued to protectionremoval by dissolution in dry dioxane (200 mL) and MeOH (4 mL) followed by treatment with 4M HCl in dioxane (2 mL). After stirring 2 hours at room temperature ethyl ether was added and the precipitate was filtered and washed with ether. The title compound was obtained as a white solid (154 mg, 0.43 mmol, 75% yield).

1H NMR (400 MHz, DMSO-D6) δ ppm 2.87-2.94 (m, 2 H) 3.39-3.51 (m, 2 H) 6.01 (s, 2 H) 7.01-7.07 (m, 2 H) 7.19-7.25 (m, 1 H) 7.26-7.34 (m, 3 H) 7.39 (s, 1 H) 7.71 (s, 1 H) 8.00 (s, 2 H) 8.09 (d, J=6.83 Hz, 1 H).

By working in an analogous way as in Example 29, the following compound in Examples 30-51 were also obtained:

### EXAMPLE 30

### 2-Pyridin-4-yl-1-[2-(tetrahydro-pyran-2-yloxy)-ethyl]-1,5,6,7-tetrahydro-pyrrolo[3,2-c]pyridin-one

1H NMR (400 MHz, DMSO-D6) δ ppm 1.27-1.56 (m, 6 H) 2.92 (t, J=6.83 Hz, 2 H) 3.29-3.40 (m, 1 H) 3.44 (ddd, J=6.95, 2.56 Hz, 1 H) 3.47-3.52 (m, 2 H) 3.64-3.71 (m, 2 H) 4.29 (t, J=5.30 Hz, 2 H) 4.40-4.43 (m, 1 H) 6.61 (s, 1 H) 7.09 (s, 1 H) 7.52 (s, 1 H) 7.54 (s, 1 H) 8.58-8.59 (m, 1 H) 8.59-8.60 (m, 1 H).

Removal of the protecting group was achieved by treating with 2N HCl at 40° C. for 1 hour:

### EXAMPLE 31

### 1-(2-Hydroxy-ethyl)-2-pyridin-4-yl-1,5,6,7-tetrahydro-pyrrolo[3,2-c]pyridin-4-one

1H NMR (400 MHz, DMSO-D6) δ ppm 2.97 (t, J=6.89 Hz, 2 H) 3.28-3.53 (m, 2 H) 3.65 (t, J=5.43 Hz, 2 H) 4.25 (t, J=5.49 Hz, 2 H) 7.13-7.18 (m, 1 H) 7.26-7.31 (m, 1 H) 8.12-8.16 (m, J=6.22 Hz, 2 H) 8.74 (s, 1 H) 8.76 (s, 1 H).

### EXAMPLE 32

### 2-(4Oxo-2-pyridin-4-yl-4,5,6,7-tetrahydro-pyrrolo[3,2-c]pyridin-1-yl)-acetamide

1H NMR (400 MHz, DMSO-D6) δ ppm 2.96 (t, J=6.77 Hz, 2 H) 3.46 (ddd, J=6.86, 2.26 Hz, 2 H) 5.18 (s, 2 H) 7.33 (s, 1 H) 7.62 (d, J=2.32 Hz, 1 H) 7.65 (s, 1 H) 7.99 (s, 1 H), 8.19 (s, 1 H) 8.21 (s, 1 H) 8.66 (s, 1 H) 8.68 (s, 1 H) 12.71 (s, 1 H).

### EXAMPLE 33

### 4-Oxo-2-pyridin-4-yl-4,5,6,7-tetrahydro-pyrrolo[3,2-c]pyridine-1-carboxylic acid methyl ester

1H NMR (400 MHz, DMSO-D6) δ ppm 3.12 (t, J=6.95 Hz, 2 H) 3.46 (ddd, J=6.98, 2.50 Hz, 2 H) 3.82 (s, 3 H) 6.72 (s, 1 H) 7.41 (d, J=6.22 Hz, 3 H) 8.56 (d, J=6.10 Hz, 2 H).

### EXAMPLE 34

### 1-Ethyl-2-pyridin-4-yl-1,5,6,7-tetrahydro-pyrrolo[3,2-c]pyridin-4-one

1H NMR (400 MHz, DMSO-D6) δ ppm 1.28 (t, J=7.19 Hz, 3 H) 2.97 (t, J=6.83 Hz, 2 H) 3.11-3.30 (m, 2 H) 4.21 (q, J=7.19 Hz, 2 H) 7.18 (s, 1 H) 7.29 (s, 1 H) 8.00 (s, 1 H) 8.01 (s, 1 H) 8.74 (s, 1 H) 8.76 (s, 1 H).

### EXAMPLE 35

### 2-Pyridin-4-yl-1-(2,2,2-trifluoro-ethyl)-1,5,6,7-tetrahydro-pyrrolo[3,2-c]pyridin-4-one

1H NMR (400 MHz, DMSO-D6) δ ppm 2.98 (t, J=6.71 Hz, 2 H) 3.41-3.55 (m, 2 H) 5.22 (q, J=8.86 Hz, 2 H) 7.04 (s, 1 H) 7.39 (s, 1 H) 7.97 (s, 2 H) 8.77 (s, 1 H) 8.79 (s, 1 H).

### EXAMPLE 36

### 2-(3-Fluoro-pyridin-4-yl)-1-(2,2,2-trifluoro-ethyl)-1,5,6,7-tetrahydro-pyrrolo[3,2-c]pyridin-4-one

1H NMR (400 MHz, DMSO-D6) δ ppm 2.90-2.97 (m, 2 H) 4.89-4.99 (m, 2 H) 6.63 (s, 1 H) 7.29 (s, 1 H) 7.54-7.59 (m, 1 H) 8.51-8.55 (m, 1 H) 8.71 (s,1 H).

### EXAMPLE 37

### 3-Methyl-2-pyridin-4-yl-1-(2,2,2-trifluoro-ethyl)-1,5,6,7-tetrahydro-pyrrolo[3,2-c]pyridin-4-one

1H NMR (400 MHz, DMSO-D6) δ ppm 2.14 (s, 3 H) 2.83-2.90 (m, 2 H) 3.37-3.44 (m, 2 H) 4.81-4.92 (m, 2 H) 7.12 (s, 1 H) 7.32-7.37 (m, 2 H) 8.63-8.68 (m, 2 H).

### EXAMPLE 38

### 2-(2-Amino-pyrimidin-4-yl)-1-methyl-1,5,6,7-tetrahydro-pyrrolo[3,2-c]pyridin-4-one

1H NMR (400 MHz, DMSO-D6) δ ppm 2.83-2.89 (m, 2 H) 3.40-3.47 (m, 2 H) 3.96 (s, 3 H) 6.54 (s, 2 H) 6.89 (d, 1 H, J=5.37 Hz) 7.01 (s, 1 H) 7.09 (s, 1 H) 8.14 (d, 1 H, J=5.37 Hz).

### EXAMPLE 39

### 2-(2-Amino-pyrimidin-4-yl)-1-(2,2,2-trifluoro-ethyl)-1,5,6,7-tetrahydro-pyrrolo[3,2-c]pyridin-4-one

1H NMR (400 MHz, DMSO-D6) δ ppm 3.01 (t, J=6.70 Hz, 2 H) 3.43-3.51 (m, 2 H) 5.89 (d, J=7.76 Hz, 2 H) 7.40 (d, J=6.70 Hz, 1 H) 7.50 (s, 1 H) 7.74 (s, 1 H) 8.23 (d, J=6.55 Hz, 1 H).

### EXAMPLE 40

### 2-(2-Amino-pyrimidin-4-yl)-1-ethyl-1,5,6,7-tetrahydro-pyrrolo[3,2-c]pyridin-4-one

1H NMR (400 MHz, DMSO-D6) δ ppm 1.29 (t, J=7.08 Hz, 3 H) 2.99 (t, J=6.78 Hz, 2 H) 3.48 (t, J=6.77 Hz, 2 H) 4.62 (q, J=7.00 Hz, 2 H) 7.38 (s, 1 H) 7.40 (d, J=7.00 Hz, 1 H) 7.72 (s, 1 H) 8.16 (d, J=7.00 Hz, 1 H).

### EXAMPLE 41

### 2-(2-Amino-pyrimidin-4-yl)-1-(4,4,4-trifluoro-butyl)-1,5,6,7-tetrahydro-pyrrolo[3,2-c]pyridin-4-one

1H NMR (400 MHz, DMSO-D6) δ ppm 1.80-1.90 (m, 2 H) 2.35-2.49 (m, 2 H) 2.96 (t, J=6.77 Hz, 2 H) 3.47 (ddd, J=6.80, 2.26 Hz, 2 H) 4.64 (t, J=7.32 Hz, 2 H) 7.35 (d, J=6.71 Hz 1 H) 7.36 (s, 1 H) 7.68 (s, 1 H) 7.94-8.04 (m, 2 H) 8.13 (d, J=6.71 Hz, 1 H).

### EXAMPLE 42

### 2-(2-Amino-pyrimidin-4-yl)-1-(4-trifluoromethyl-benzyl)-1,5,6,7-tetrahydro-pyrrolo]3,2-c]pyridin-4-one

1H NMR (400 MHz, DMSO-D6) δ ppm 2.85-2.93 (m, 2 H) 3.42-3.54 (m, 2 H) 6.10 (s, 2 H) 7.22-7.27 (m, 2 H) 7.32 (d, 1 H, J=6.58 Hz) 7.41 (s, 1 H) 7.64-7.70 (m, 2 H) 7.73 (s, 1 H) 8.09 (d, 1 H, J=6.70 Hz).

### EXAMPLE 43

### 2-(2-Amino-pyrimidin-4-yl)-1-propyl-1,5,6,7-tetrahydro-pyrrolo[3,2-c]pyridin-4-one

1H NMR (400 MHz, DMSO-D6) δ ppm 0.85 (t, 3 H, J=7.32 Hz) 1.57-1.70 (m, 2 H) 2.91-3.00 (m, 2 H) 3.42-3.52 (m, 2 H) 4.56 (t, 2 H, J=7.19Hz) 7.31-7.39 (m, 2 H) 7.65 (s, 1 H) 8.12 (d, 1 H, J=6.82 Hz).

### EXAMPLE 44

### 2-(2-Amino-pyrimidin-4-yl)-1-butyl-1,5,6,7-tetrahydro-pyrrolo[3,2-c]pyridin-4-one

1H NMR (400 MHz, DMSO-D6) δ ppm 0.87 (t, 3 H, J=7.44 Hz) 1.22-1.34 (m, 2 H) 1.52-1.63 (m, 2 H) 2.91-2.99 (m, 2 H) 3.42-3.51 (m, 2 H) 4.60 (t, 2 H, J=7.19 Hz) 7.32 (d, 1H, J=6.95 Hz) 7.34 (s, 1 H) 7.61 (s, 1 H) 7.97 (s, 2 H) 8.12 (d, 1 H, J=6.70 Hz).

### EXAMPLE 45

### 2-(2-Amino-pyrimidin-4-yl)-1-isobutyl-1,5,6,7-tetrahydro-pyrrolo[3,2-c]pyridin-4-one

1H NMR (400 MHz, DMSO-D6) δ ppm 0.82 (d, J=6.71 Hz, 6 H) 1.80-1.94 (m, 1 H) 2.88-2.98 (m, 2 H) 3.42-3.54 (m, 2 H) 4.43 (d, J=7.20 Hz, 2 H) 7.32 (d, J=6.83 Hz, 1 H) 7.36 (s, 1 H) 7.62 (s, 1 H) 7.97 (s, 2 H) 8.12 (d, J=6.71 Hz, 1 H).

### EXAMPLE 46

### 2-(2-Amino-pyrimidin-4-yl)-1-(2-hydroxy-ethyl)-1,5,6,7-tetrahydro-pyrrolo[3,2-c]pyridin-4-one

1H NMR (400 MHz, DMSO-D6) δppm 2.97 (t, J=6.77 Hz, 2 H) 3.36-3.50 (m, 2 H) 3.66 (t, J=5.37 Hz, 2 H) 4.58 (t, J=5.30 Hz, 2 H) 7.33 (d, J=10.12 Hz, 1 H) 7.35 (d, J=7.19 Hz, 1 H) 7.69 (s, 1 H) 8.09 (d, J=6.95 Hz, 1 H).

### EXAMPLE 47

### 2-(2-Amino-pyrimidin-4-yl)-6-benzyl-1-ethyl-1,5,6,7-tetrahydro-pyrrolo[3,2-c]pyridin-4-one

1H NMR (400 MHz, DMSO-D6) δ ppm 1.21 (d, J=6.95 Hz, 3 H) 2.66-3.02 (m, 4 H) 3.94-4.05 (m, 1 H) 4.40-4.60 (m, 2 H) 7.23-7.37 (m, 6 H) 7.62 (s, 1 H) 8.00 (bs, 2H) 8.12 (d, J=6.83 Hz, 1 H).

### EXAMPLE 48

### 2-(2-Amino-pyrimidin-4-yl)-1,7-diethyl-1,5,6,7-tetrahydro-pyrrolo[3,2-c]pyridin-4-one

1H NMR (400 MHz, DMSO-D6) δ ppm 0.99 (t, J=7.38 Hz, 3 H) 1.28 (t, J=6.95 Hz, 3 H) 1.48-1.68 (m, 2 H) 2.95-3.02 (m, 1 H) 3.23-3.59 (m, 2 H) 4.33-4.44 (m, 1 H) 4.70-4.81 (m, 1 H) 7.25 (d, J=4.63 Hz, 1 H) 7.32 (d, J=6.83 Hz, 1 H) 7.61 (s, 1 H) 8.04 (bs, 3 H) 8.12 (d, J=6.71 Hz, 1 H).

### EXAMPLE 49

### 2-(2-Amino-pyrimidin-4-yl)-7-ethyl-4-oxo-1-(2,2,2-trifluoro-ethyl)-1,4,6,7-tetrahydro-pyrrolo[3,2-c]pyridine-5-carboxylic acid tert-butyl ester

The racemate, as Boc derivative, was subjected to chiral separation so to obtain the pure enantiomers. Chiral chromatography was performed on Chiralpack® AD column (5×50 cm). Mobile phase was nHexane/Ethanol/Methanol 75/8/17.

Analytical Conditions:

Chiralpack® AD column with precolumn, mobile phase nHexane//Ethanol/Methanol 80/5/15.

### EXAMPLE 50

### (R and S)-2-(2-Amino-pyrimidin-4-yl)-7-ethyl-4oxo-1-(2,2,2-trifluoro-ethyl)-1,4,6,7-tetrahydro-pyrrolo[3,2-c]pyridine-5-carboxylic acid tert-butyl ester

e.e.>99%

The enantiomeric Boc derivatives were deprotected as usual in TFA yielding the two final enantiomers:

### EXAMPLE 51

### (R and S)-2-(2-Amino-pyrimidin-4-yl)-7-ethyl-1-(2,2,2-trifluoro-ethyl)-5,6,7-tetrahydro-pyrrolo[3,2-c]pyridin-4-one

1H NMR (400 MHz, DMSO-D6) δ ppm 0.97 (t, J=7.38 Hz, 3 H) 1.54-1.63 (m, J=7.32 Hz, 2 H) 2.99-3.07 (m, 1 H) 3.36-3.44 (m, 1 H) 3.46-3.54 (m, 1 H) 5.15-5.29 (m, 1 H) 6.32-6.45 (m, 1 H) 7.35 (d, J=6.46 Hz, 1 H) 7.39 (d, J=4.51 Hz, 1 H) 7.65-7.69 (m, 1 H) 8.12 (bs, 3 H) 8.22 (d, J=6.58 Hz, 1 H).

### EXAMPLE 52

### 2-(2-Amino-pyrimidin-4-yl)-1-(2-fluoro-ethyl)-1,5,6,7-tetrahydro-pyrrolo[3,2-c]pyridin-4-one

A mixture of 2-(2-amino-pyrimidin-4-yl)-4-oxo-1,4,6,7-tetrahydro-pyrrolo[3,2-c]pyridine-5-carboxylic acid tert-butyl ester (99.2 mg, 0.3 mmol), 1-bromo-2-fluoro ethane (90 □M, 1.2 mmol), potassium carbonate (166 mg, 1.2 mmol) in anhydrous DMF (2 mL) was stirred under heating at 65° C. for 4 hours. After cooling the reaction mixture was treated with water and ethyl acetate, the organic layer was extracted with brine and then dried over anhydrous sodium sulphate. The crude product was purified by flash chromatography over silica gel (eluant DCM/EtOH 95:5). Pure 2-(2-amino-pyrimidin-4-yl)-1-(2-fluoro-ethyl)-4-oxo-1,4,6,7-tetrahydro-pyrrolo[3,2-c]pyridine-5-carboxylic acid tert-butyl ester, obtained as a solid (90 mg, 0.24 mmol, 80% yield), was subdued to deprotection by dissolving it in dry MeOH (5 mL) and 4M HCl in dioxane (0.6 mL) and stirring the solution at room temperature for 3 hours. After concentration the precipitate was filtered off, washed with ether and dried. Obtained the title compound as a white solid (71 mg, 95% yield).

1H NMR (400 MHz, DMSO-D6) δ ppm 2.91-2.98 (m, 2 H) 3.40-3.50 (m, 2 H) 4.67-4.75 (m, 1 H) 4.80-4.85 (m, 1 H) 4.85-4.90 (m, 1 H) 4.90-4.97 (m, 1 H) 7.40 (d, J=6.83 Hz, 1 H and 1 singlet, 1H) 7.76 (s, 1 H) 8.14 (d, J=6.82 Hz, 1 H) 8.17-8.51 (m, 2 H).

By working in an analogous way as in Example 52, the following compounds in Examples 53-59 were also obtained:

### EXAMPLE 53

### 2-(2-Amino-pyrimidin-4-yl)-1-(2,2,2-trifluoro-ethyl)-1,5,6,7-tetrahydro-pyrrolo[3,2-c]pyridin-4-one (alternative synthesis)

1H NMR (400 MHz, DMSO-D6) δ ppm 3.01 (t, J=6.70 Hz, 2 H) 3.43-3.51 (m, 2 H) 5.89 (d, J=7.76 Hz, 2 H) 7.40 (d, J=6.70 Hz, 1 H) 7.50 (s, 1 H) 7.74 (s, 1 H) 8.23 (d, J=6.55 Hz, 1 H).

### EXAMPLE 54

### 2-(2-Amino-pyrimidin-4-yl)-6-isobutyl-1-(2,2,2-trifluoro-ethyl)-1,5,6,7-tetrahydro-pyrrolo[3,2-c]pyridin-4-one

1H NMR (400 MHz, DMSO-D6) δ ppm 0.88 (dd, J=10.61, 6.58 Hz, 6 H) 1.30-1.42 (m, 1 H) 1.43-1.54 (m, 1 H) 1.71-1.83 (m, 1 H) 2.62-2.71 (m, 1 H) 3.12-3.21 (m, 1 H) 3.65-3.75 (m, 1 H) 5.60-5.84 (m, 1 H) 5.93-6.10 (m, 1 H) 7.29 (d, J=6.46 Hz, 1 H) 7.42 (s, 1 H) 7.61 (s, 1 H) 7.94 (s, 2 H) 8.17 (d, J=6.58 Hz, 1 H).

### EXAMPLE 55

### 2-(2-Amino-pyrimidin-4-yl)-1-isopropyl-1,5,6,7-tetrahydro-pyrrolo[3,2-c]pyridin-4-one

1H NMR (400 MHz, DMSO-D6) δ ppm 1.50 (d, J=6.95 Hz, 6 H) 3.05-3.14 (m, 2 H) 3.39-3.48 (m, 2 H) 5.85-5.99 (m, 1 H) 7.27 (d, J=6.71 Hz, 1 H) 7.36 (s, 1 H) 7.44 (s, 1 H) 7.99 (s, 2 H) 8.14 (d, J=6.70 Hz, 1 H).

### EXAMPLE 56

### 2-(2-Amino-pyrimidin-4-yl)-6,6-dimethyl-1-(2,2,2-trifluoro-ethyl)-1,5,6,7-tetrahydro-pyrrolo[3,2-c]pyridin-4-one

1H NMR (400 MHz, DMSO-D6) δ ppm 1.27 (s, 6 H) 2.96 (s, 2 H) 5.85 (d, J=6.83 Hz, 2 H) 7.30 (s, 1 H) 7.43 (s, 1 H) 7.63 (s, 1 H) 7.94 (bs, 3 H) 8.18 (d, J=6.46 Hz, 1 H).

### EXAMPLE 57

### 2-(2-Amino-pyrimidin-4-yl)-7-methyl-1-(2,2,2-trifluoro-ethyl)-1,5,6,7-tetrahydro-pyrrolo[3,2-c]pyridin-4-one

1H NMR (400 MHz, DMSO-D6) δ ppm 1.24 (d, J=6.83 Hz, 3 H) 3.20-3.31 (m, 1 H) 3.41-3.64 (m, 2 H) 5.25-5.40 (m, 1 H) 6.22-6.36 (m, 1 H) 7.37 (d, J=6.71 Hz, 1 H) 7.45 (d, J=4.51 Hz, 1 H) 7.70 (s, 1 H) 8.23 (d, J=6.71 Hz, 1 H).

### EXAMPLE 58

### (R and S)-2-(2-Amino-pyrimidin-4-yl)-7-isopropyl-1-(2,2,2-trifluoro-ethyl)-1,5,6,7-tetrahydro-pyrrolo[3,2-c]pyridin-4-one

1H NMR (400 MHz, DMSO-D6) δ ppm 0.91 (d, J=6.95 Hz, 3 H) 0.96 (d, J=6.71 Hz, 3 H) 1.85-1.97 (m, 1 H) 2.89 (s, 1 H) 3.34-3.53 (m, 2 H) 5.01-5.15 (m, 1 H) 6.43-6.58 (m, 1 H) 7.33 (d, J=6.95 Hz, 1 H) 7.38 (d, J=4.27 Hz, 1 H) 7.66 (s, 1 H) 8.20 (bs, 3 H) 8.20 (d, J=6.46 Hz, 1 H).

### EXAMPLE 59

### 2-(2-Amino-pyrimidin-4-yl)-1-cyclopropylmethyl-1,5,6,7-tetrahydro-pyrrolo[3,2-c]pyridin-4-one

A mixture of 1-(2-aminopyrimidin-4-yl)-2-bromoethanone hydrobromide (0.9 g, 3 mmol), cyclopropylmethylamine (0.85 g, 12 mmol, 1.03 mL) and piperidin-2,4-dione (0.51 g, 4.5 mmol), dissolved in absolute ethanol (10 mL), was stirred under heating at 70° C. for 3 hours in a glass pressure tube. Ethanol was evaporated off and the crude reaction mixture was purified by flash chromatography on silica gel (eluant: DCM/MeOH 4:1) and then by crystallization from MeOH. The title compound was obtained as white solid (0.42 g, 49% yield).

1H NMR (400 MHz, DMSO-D6) δ ppm 0.30-0.48 (m, 4 H) 1.12-1.26 (m, 1 H) 2.97 (t, J=6.77 Hz, 2 H) 3.45 (t, J=6.46 Hz, 2 H) 4.61 (d, J=7.07 Hz, 2 H) 7.34-7.44 (d, J=6.95Hz, 1 H and one s, 1H) 7.71 (s, 1 H) 8.15 (d, J=6.95 Hz, 1 H) 8.31 (s, 1 H).

By working in an analogous way as in Example 59, the following compounds in Example 60-68 were also obtained:

### EXAMPLE 60

### 1-Methyl-2-pyridin-4-yl-1,5,6,7-tetrahydro-pyrrolo[3,2-c]pyridin-4-one

1H NMR (400 MHz, DMSO-D6) δ ppm 2.87 (t, J=6.89 Hz, 1 H) 3.45 (td, J=6.95, 2.56 Hz, 2 H) 3.65 (s, 3 H) 6.66 (s, 1 H) 7.06 (m, 1 H) 7.50 (d, J=6.10 Hz, 2 H) 8.59 (d, J=6.22 Hz, 2 H).

### EXAMPLE 61

### 2-(2-Amino-pyrimidin-4-yl)-1-phenyl-1,5,6,7-tetrahydro-pyrrolo[3,2-c]pyridin-4-one

1 H NMR (400 MHz, DMSO-D6) δ ppm 2.90-2.99 (m, 2 H) 3.48-3.58 (m, 2 H) 7.54-7.68 (m, 7 H) 8.36-8.75 (m, 4 H).

### EXAMPLE 62

### 2-(2-Amino-pyrimidin-4-yl)-1-cyclopropyl-1,5,6,7-tetrahydro-pyrrolo[3,2-c]pyridin-4-one

1H NMR (400 MHz, DMSO-D6) δ ppm 0.74-0.82 (m, 2 H) 1.08-1.17 (m, 2 H) 2.99 (t, J=6.71 Hz, 2 H) 213.40-3.47 (m, 2 H) 3.53-3.60 (m, 1 H) 7.30 (d, 1 H, J=6.83 Hz) 7.32 (s, 1 H) 7.40 (s, 1 H) 8.03 (bs, 2 H) 8.18 (d, 1 H, J=6.71 Hz).

### EXAMPLE 63

### 2-(2-Amino-pyrimidin-4-yl)-1-(1-methyl-piperidin-4-yl)-1.5.6,7-tetrahydro-pyrrolo[3,2-c]pyridin-4-one

1H NMR (400 MHz, DMSO-D6) δ ppm 2.07-2.18 (m, 4 H) 2.79 (d, J=4.63 Hz, 3 H) 3.12-3.20 (m, 2 H) 3.21-3.54 (m, 6 H) 5.66-5.86 (m, 1 H) 7.27 (s, 1 H) 7.41 (s, 1 H) 7.46 (s, 1 H) 8.07 (bs, 2 H) 8.16 (d, J=6.46 Hz, 1 H) 10.45-10.71 (m, 1H).

### EXAMPLE 64

### 2-(2-Amino-pyrimidin-4-yl)-1-cyclohexyl-1,5,6,7-tetrahydro-pyrrolo[3,2-c]pyridin-4-one

1H NMR (400 MHz, DMSO-D6) δ ppm 1.15-2.04 (m, 10 H) 2.98-3.07 (m, 2 H) 3.52-3.61 (m, 2 H) 4.04-4.17 (m, 1 H) 7.40-7.49 (m, 2 H) 8.36-8.51 (m, 5 H) 8.58-8.65 (m, 1 H).

### EXAMPLE 65

### 2-(2-Amino-pyrimidin-4-yl)-7,7-dimethyl-1-(2,2,2-trifluoro-ethyl)-1,5,6,7-tetrahydro-pyrrolo[3,2-c]pyridin-4-one

1H NMR (400 MHz, DMSO-D6) δ ppm 1.39 (s, 6 H) 3.26-3.38 (m, 2 H) 5.16-5.28 (m, 2 H) 7.44 (s, 1 H) 8.03-8.93 (m, 5 H).

### EXAMPLE 66

### 2-(2-Amino-pyrimidin-4-yl)-1-cyclopropylmethyl-7,7-dimethyl-1,5,6,7-tetrahydro-pyrrolo[3,2-c]pyridin-4-one

1H NMR (400 MHz, DMSO-D6) δ ppm 0.27-0.44 (m, 4 H) 1.03-1.15 (m, 1 H) 1.44 (s, 6 H) 3.15-3.21 (m, 2 H) 4.79 (d, J=6.82 Hz, 2 H) 7.28 (d, J=6.21 Hz, 1 H) 7.45-7.55 (m, 2 H) 8.17 (d, J=6.71 Hz, 1 H) 8.05 (bs, 1 H).

### EXAMPLE 67

### 2-(2-Amino-pyrimidin-4-yl)-1-cyclobutyl-1,5,6,7-tetrahydro-pyrrolo[3,2-c]pyridin-4-one

1H NMR (400 MHz, DMSO-D6) δ ppm 1.68-1.89 (m, 2 H) 2.38-2.59 (m, 3 H) 3.11-3.21 (m, 2 H) 3.24-3.63 (m, 2 H) 5.66-5.82 (m, 1 H) 7.30 (d, J=6.71 Hz, 1 H) 7.42 (s, 1 H) 7.46 (s, 1 H) 8.16 (d, J=6.83 Hz, 1 H) 8.26 (bs, 2 H).

### EXAMPLE 68

### 2-(2-Amino-pyrimidin-4-yl)-6-benzyl-1-(2,2,2-trifluoro-ethyl)-1,5,6,7-tetrahydro-pyrrolo[3,2-c]pyridin-4-one

1H NMR (400 MHz, DMSO-D6) δ ppm 2.74 (dd, J=16.78, 8.54 Hz, 1 H) 2.84 (dd, J=13.73, 7.63 Hz, 1 H) 2.95 (dd, J=13.57, 5.34 Hz, 1 H) 3.01 (dd, J=1 6.93, 5.34 Hz, 1 H) 3.95-4.05 (m, 1 H) 5.64-5.80 (m, 1 H) 5.81-6.00 (m, 1 H) 7.21-7.29 (m, 3 H) 7.29-7.36 (m, 3 H) 7.42 (s, 1 H) 7.65 (s, 1 H) 8.18 (d, J=6.41 Hz, 1 H) 8.18 (d, J=6.41 Hz, 1 H).

### EXAMPLE 69

### 2-(2-Amino-pyrimidin-4-yl)-3-iodo-1-methyl-4-oxo-1,4,6,7-tetrahydro-pyrrolo]3,2-c]pyridine-5-carboxylic acid tert-butyl ester

To a solution of 2-(2-amino-pyrimidin-4-yl)-1-methyl-4-oxo-1,4,6,7-tetrahydro-pyrrolo[3,2-c]pyridine-5-carboxylic acid tert-butyl ester (70 mg, 0.2 mmol) in anhydrous DMF (1 mL) a solution of N-iodosuccinimide (45 mg, 0.2 mmol) in anh. DMF (1 mL) was dropped at room temperature. After stirring overnight the reaction mixture was poured in ethylacetate and water, the organic layer was washed with saturated aqueous sodium bisulphite, then with water, dried over anhydrous sodium sulphate and concentrated to yield the title compound in 80% yield.

ESI (+) MS: m/z 470 (MH+).

Based on the procedures described above, the following compounds can be also prepared:
2-(2-Amino-pyrimidin-4-yl)-1-(1-phenyl-ethyl)-1,5,6,7-tetrahydro-pyrrolo[3,2-c]pyridin-4-one;
2-(2-Amino-pyrimidin-4-yl)-7-cyclobutyl-1-ethyl-1,5,6,7-tetrahydro-pyrrolo[3,2-c]pyridin-4-one;
2-(2-Amino-pyrimidin-4-yl)-7-cyclobutyl-1-(2,2,2-trifluoro-ethyl)-1,5,6,7-tetrahydro-pyrrolo[3,2-c]pyridin-4-one;
2-(2-Amino-pyrimidin-4-yl)-7-cyclobutyl-1-propyl-1,5,6,7-tetrahydro-pyrrolo[3,2-c]pyridin-4-one;
2-(2-Amino-pyrimidin-4-yl)-7-cyclobutyl-1-isobutyl-1,5,6,7-tetrahydro-pyrrolo[3,2-c]pyridin-4-one;
2-(2-Amino-pyrimidin-4-yl)-7-cyclobutyl-1-cyclopropylmethyl-1,5,6,7-tetrahydro-pyrrolo[3,2-c]pyridin-4-one;
2-(2-Amino-pyrimidin-4-yl)-1,7-dicyclobutyl-1,5,6,7-tetrahydro-pyrrolo[3,2-c]pyridin4-one;
2-(2-Amino-pyrimidin-4-yl)-7-cyclobutyl-1-cyclobutylmethyl-1,5,6,7-tetrahydro-pyrrolo[3,2-c]pyridin-4-one;
2-(2-Amino-pyrimidin-4-yl)-3-iodo-1-methyl-1,5,6,7-tetrahydro-pyrrolo[3,2-c]pyridin-4-one;
2-(2-Amino-pyrimidin-4-yl)-6-cyclopropyl-1-ethyl-1,5,6,7-tetrahydro-pyrrolo[3,2-c]pyridin-4-one;
2-(2-Amino-pyrimidin-4-yl)-6-cyclopropyl-1-(2,2,2-trifluoro-ethyl)-1,5,6,7-tetrahydro-pyrrolo[3,2-c]pyridin-4-one;
2-(2-Amino-pyrimidin-4-yl)-6-cyclopropyl-1-propyl-1,5,6,7-tetrahydro-pyrrolo[3,2-c]pyridin-4-one;
2-(2-Amino-pyrimidin-4-yl)-6-cyclopropyl-1-isobutyl-1,5,6,7-tetrahydro-pyrrolo[3,2-c]pyridin-4-one;
2-(2-Amino-pyrimidin-4-yl)-6-cyclopropyl-1-cyclopropylmethyl-1,5,6,7-tetrahydro-pyrrolo[3,2-c]pyridin-4-one;
2-(2-Amino-pyrimidin-4-yl)-1-cyclobutyl-6-cyclopropyl-1,5,6,7-tetrahydro-pyrrolo[3,2-c]pyridin-4-one; and
2-(2-Amino-pyrimidin-4-yl)-1-cyclobutylmethyl-6-cyclopropyl-1,5,6,7-tetrahydro-pyrrolo[3,2-c]pyridin-4-one.

### EXAMPLE 70

### 2-(2-Amino-pyrimidin-4-yl)-1-(1-phenyl-ethyl)-1,5,6,7-tetrahydro-pyrrolo[3,2-c]pyridin4-one

¹H NMR (400 MHz, DMSO-D6) δ ppm 1.87 (d, J=7.07 Hz, 3 H) 1.89-2.01 (m, 1 H) 2.64-2.79 (m, 1 H) 3.02-3.13 (m, 1 H) 3.26-3.29 (m, 1 H) 7.15 (d, J=7.92 Hz, 2 H) 7.25-7.33 (m, 2 H) 7.33-7.38 (m, 2 H) 7.39-7.42 (m, 1 H) 7.48 (q, J=7.44 Hz, 1 H) 7.70-8.71 (bs, 3 H) 8.15 (d, J=6.46 Hz, 1 H).

### EXAMPLE 71

### 1-(2-Methoxy-ethyl)-2-pyridin-4-yl-1,5,6,7-tetrahydro-pyrrolo[3,2-c]pyridin-4-one

To a solution of 114 μL (1.44 mmol) of 2-methoxy-ethanol and 377 mg of triphenylphosphine (1.44 mmol) in dry THF (0.5 mL) at 0° C. 224 μL of diethyl-azo-dicarboxylate (1.44 mmol) were added. The mixture was kept at room temperature for 15 minutes and a solution of 150 mg (0.48 mmol) of tert-butyl 4-oxo-2-pyridin-4-yl-1,4,6,7-tetrahydro-pyrrolo[3,2-c]pyridine-5-carboxylate in dry DMF (2.3 mL) was added. The mixture was heated at 65° C. for 90 min and the solvent was removed under vacuum. Purification of the crude material by flash chromatography with dichloromethane/methanol/acetone 90/3/7 gave 97 mg of tert-butyl 1-(2-methoxy-ethyl)-4-oxo-2-pyridin-4-yl-1,4,6,7-tetrahydro-pyrrolo[3,2-c]pyridine-5-carboxylate (54% yield). This product was treated with TFA/DCM 1/1 (3mL) at rt for 1 hour then the solvent was removed under vacuum. The residue was dissolved with methanol and an excess of HCl 1.25 M in methanol was added. The solvent was removed under vacuum and the residue was treated with diethyl ether. The precipitate was filtered, washed and dried under vacuum thus affording 50mg of the title compound (62% yield).

1H NMR (400 MHz, DMSO-D6) δ ppm 2.95 (t, J=6.93 Hz, 2 H) 3.15 (s, 3 H) 3.38-3.48 (m, 2 H) 3.55 (t, J=5.15 Hz, 2 H) 4.35 (t, J=5.15 Hz, 2 H) 7.13 (s, 1 H) 7.28 (s, 1 H) 8.13 (s, 2 H) 8.81 (s, 2 H)

It is to be understood that many modifications and variations may be devised given the above description of the principles of the invention. It is intended that all such modifications and variations can be considered as within the spirit and scope of this invention, as it is defined in the following claims.

We claim:

1. A compound represented by formula (I) 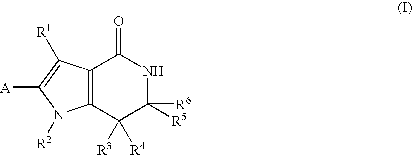
wherein
A is selected from the group consisting of pyridin-4-yl, 3-fluoro-pyridin-4-yl, and 2-amino-pyrimidin-4-yl;
R¹ is selected from the group consisting of hydrogen, halogen and (C₁-C₆)alkyl group;
R² is selected from the group consisting of (C₁-C₆)alkyl, (C₁-C₆)alkenyl, (C₁-C₆)alkynyl, (C₃-C₆)cycloalkyl, (C₁-C₆)haloalkyl, (C₁-C₆)polyfluorinated alkyl, heterocyclyl, aryl, heteroaryl, (C₃-C₆)cycloalkyl-(C₁-C₆)alkyl, heterocyclyl-(C₁-C₆)alkyl, aryl-(C₁-C₆)alkyl, heteroaryl-(C₁-C₆)alkyl, (C₁-C₈)hydroxyalkyl, (C₁-C₈)alkoxy-(C₁-C₈)alkyl, aryloxy-(C₁-C₈)alkyl, heteroaryloxy-(C₁-C₈)alkyl, (C₁-C₈)aminoalkyl, (C₁-C₈)alkylamino-(C₁-C₈)alkyl, (C₁-C₈)dialkylamino-(C₁-C₈)alkyl, carbamoyl(C₁-C₈)alkyl, and alkoxycarbonyl, wherein each of said aryl, heteroaryl, heterocyclyl, aryloxy, or heteroaryloxy moieties can be unsubstituted or substituted by one or substituents, each substituent being independently selected from the group consisting of alkyl, aryl, —OCF₃, —OC(O)alkyl, —OC(O)aryl, —CF₃, heteroaryl, aralkyl, alkylaryl, heteroaralkyl, alkylheteroaryl, hydroxy, hydroxyalkyl, alkoxy, aryloxy, aralkoxy, acyl, aryl, halo, haloalkyl, haloalkoxy, nitro, cyano, carboxy, alkoxycarbonyl, aryloxycarbonyl, aralkoxycarbonyl, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, cycloalkyl, heterocyclyl, heterocyclenyl, —NH(alkyl), —NH(cycloalkyl), and —N(alkyl)₂;
R³, R⁴, R⁵ and R⁶ are each independently selected from the group consisting of hydrogen, (C₁-C₆)alkyl, (C₃-C₆)cycloalkyl, heterocyclyl, aryl, cyloalkyl-(C₁-C₆)alkyl, heterocyclyl-(C₁-C₆)alkyl and aryl-(C₁-C₆)alkyl, wherein each of said aryl or heterocyclyl moieties can be unsubstituted or substituted by one or substituents, each being independently selected from the group consisting of alkyl, aryl, —OCF₃, —OC(O)alkyl, —OC(O)aryl, —CF₃, heteroaryl, aralkyl, alkylaryl, heteroaralkyl, alkylheteroaryl, hydroxy, hydroxyalkyl, alkoxy, aryloxy, aralkoxy, acyl, aryl, halo, haloalkyl, haloalkoxy, nitro, cyano, carboxy, alkoxycarbonyl, aryloxycarbonyl, aralkoxycarbonyl, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, cycloalkyl, heterocyclyl, heterocyclenyl, —NH(alkyl), —NH(cycloalkyl), and —N(alkyl)₂; or
R³ and R⁴ or R⁵ and R⁶, taken together, form a (C₃-C₆)cycloalkyl;
or a pharmaceutically acceptable salt or solvate thereof,
provided that the compound is not:
1-butyl-2-pyridin-4-yl-1,5,6,7-tetrahydro-pyrrolo[3,2-c]pyridin-4-one;
1-benzyl-2-pyridin-4-yl-1,5,6,7-tetrahydro-pyrrolo[3,2-c]pyridin-4-one;
1-but-3-enyl-2-pyridin-4-yl-1,5,6,7-tetrahydro-pyrrolo[3,2-c]pyridin-4-one;
1-(3-methyl-butyl)-2-pyridin-4-yl-1,5,6,7-tetrahydro-pyrrolo[3,2-c]pyridin-4-one;
1-(3-phenyl-propyl)-2-pyridin-4-yl-1,5,6,7-tetrahydro-pyrrolo[3,2-c]pyridin-4-one;
or 1-(2-cyclohexyl-ethyl)-2-pyridin-4-yl-1,5,6,7-tetrahydro-pyrrolo[3,2-c]pyridin-4-one.

2. The compound according to claim 1 wherein both R³ and R⁴ are hydrogen atoms.

3. The compound according to claim 1, wherein both R⁵ and R⁶ are hydrogen atoms.

4. The compound according to claim 1, wherein A is a 2-amino-pyrimidin-4-yl group, and R¹ is a hydrogen atom.

5. A method for treating a cell proliferatve disorder or condition in a mammal comprising administering to a mammal in need of said treatment a compound represented by formula (I) 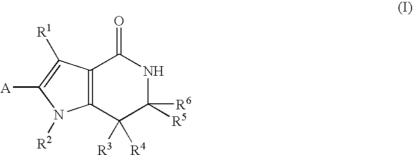
wherein
A is selected from the group consisting of pyridin-4-yl, 3-fluoro-pyridin-4-yl, and 2-amino-pyrimidin-4-yl;
R¹ is selected from the group consisting of hydrogen, halogen and (C₁-C₆)alkyl group;
R² is selected from the group consisting of (C₁-C₆)alkyl, (C₁-C₆)alkenyl, (C₁-C₆)alkynyl, (C₃-C₆)cycloalkyl, (C₁-C₆)haloalkyl, (C₁-C₆)polyfluorinated alkyl, heterocyclyl, aryl, heteroaryl, (C₃-C₆)cycloalkyl-(C₁-C₆)alkyl, heterocyclyl-(C₁-C₆)alkyl, aryl-(C₁-C₆)alkyl, heteroaryl-(C₁-C₆)alkyl, (C₁-C₈)hydroxyalkyl, (C₁-C₈)alkoxy-(C₁-C₈)alkyl, aryloxy-(C₁-C₈)alkyl, heteroaryloxy-(C₁-C₈)alkyl, (C₁-C₈)aminoalkyl, (C₁-C₈)alkylamino-(C₁-C₈)alkyl, (C₁-C₈)dialkylamino-(C₁-C₈)alkyl, carbamoyl(C₁-C₈)alkyl, and alkoxycarbonyl, wherein each of said aryl, heteroaryl, heterocyclyl, aryloxy, or heteroaryloxy moieties can be unsubstituted or substituted by one or substituents, each substituent being independently selected from the group consisting of alkyl, aryl, —OCF₃, —OC(O)alkyl, —OC(O)aryl, —CF₃, heteroaryl, aralkyl, alkylaryl, heteroaralkyl, alkylheteroaryl, hydroxy, hydroxyalkyl, alkoxy, aryloxy, aralkoxy, acyl, aryl, halo, haloalkyl, haloalkoxy, nitro, cyano, carboxy, alkoxycarbonyl, aryloxycarbonyl, aralkoxycarbonyl, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, cycloalkyl, heterocyclyl, heterocyclenyl, —NH(alkyl), —NH(cycloalkyl), and —N(alkyl)₂;
R³, R⁴, R⁵ and R⁶ are each independently selected from the group consisting of hydrogen, (C₁-C₆)alkyl, (C₃-C₆)cycloalkyl, heterocyclyl, aryl, cyloalkyl-(C₁-C₆)alkyl, heterocyclyl-(C₁-C₆)alkyl and aryl-(C₁-C₆)alkyl, wherein each of said aryl or heterocyclyl moieties can be unsubstituted or substituted by one or substituents, each being independently selected from the group consisting of alkyl, aryl, —OCF₃, —OC(O)alkyl, —OC(O)aryl, —CF₃, heteroaryl, aralkyl, alkylaryl, heteroaralkyl, alkylheteroaryl, hydroxy, hydroxyalkyl, alkoxy, aryloxy, aralkoxy, acyl, aryl, halo, haloalkyl, haloalkoxy, nitro, cyano, carboxy, alkoxycarbonyl, aryloxycarbonyl, aralkoxycarbonyl, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, cycloalkyl, heterocyclyl, heterocyclenyl, —NH(alkyl), —NH(cycloalkyl), and —N(alkyl)₂; or R³ and R⁴ or R⁵ and R⁶, taken together, form a (C₃-C₆)cycloalkyl;

6. The method according to claim 5, wherein said disorder or condition is caused by or is associated with an altered Cdc7 kinase activity.

7. A method of antagonizing activity toward Cdk2 or Cdc7, comprising administering to said Cdk2 or Cdc7 an amount of a compound of claim 1 that is effective in antagonizing activity toward Cdk2 or Cdc7.

8. A method of treating a disorder or condition in a mammal, wherein antagonist activity toward Cdk2 or Cdc7 is needed in said mammal, comprising administering to said mammal an amount of a compound of claim 1 that is effective in antagonizing activity toward Cdk2 or Cdc7.

9. A method of treating a disorder or condition in a mammal for which antagonist activity toward Cdk2 or Cdc7 is needed in said mammal, comprising administering to said mammal an amount of a compound of claim 1 that is effective in treating said disorder or condition.

10. A method according to claim 5, wherein antagonist activity toward Cdk2 or Cdc7 is needed and wherein the amount of said compound is effective in antagonizing activity toward Cdk2 or Cdc7.

11. A method according to claim 5, wherein antagonist activity toward Cdk2 or Cdc7 is needed and wherein the amount of said compound is effective in is effective in treating said disorder or condition.

12. A method of treating a disorder or condition selected from the group consisting of squamous cell carcinoma, hematopoietic tumors of myeloid or lymphoid lineage, tumors of mesenchymal origin, tumors of the central and peripheral nervous system, melanoma, seminoma, teratocarcinoma, osteosarcoma, xeroderma pigmentosum, keratoxanthoma, thyroid follicular cancer, and Kaposi's sarcoma, in a mammal, comprising administering to said mammal in need of said treatment an amount of a compound of claim 1 that is effective in treating said condition or disorder.

13. A method of treating a disorder or condition selected from the group consisting of benign prostate hyperplasia, familial adenomatosis, polyposis, neuro-fibromatosis, psoriasis, vascular smooth cell proliferation associated with atherosclerosis, pulmonary fibrosis, arthritis glomerulonephritis, post-surgical stenosis and restenosis, in a mammal, comprising administering to said mammal in need of said treatment an amount of a compound of claim 1 that is effective in treating said condition or disorder.

14. A method of treating a disorder or condition selected from the group consisting of carcinoma, squamous cell carcinoma, hematopoietic tumors of myeloid or lymphoid lineage, tumors of mesenchymal origin, tumors of the central and peripheral nervous system, melanoma, seminoma, teratocarcinoma, osteosarcoma, xeroderma pigmentosum, keratoxanthoma, thyroid follicular cancer, and Kaposi's sarcoma, in a mammal, comprising administering to said mammal in need of said treatment an amount of a compound of claim 1 that is effective in antagonizing activity toward Cdk2 or Cdc7.

15. A method of treating a disorder or condition selected from the group consisting of benign prostate hyperplasia, familial adenomatosis, polyposis, neuro-fibromatosis, psoriasis, vascular smooth cell proliferation associated with atherosclerosis, pulmonary fibrosis, arthritis glomerulonephritis, post-surgical stenosis and restenosis, in a mammal, comprising administering to said mammal in need of said treatment an amount of a compound of claim 1 that is effective in antagonizing activity toward Cdk2 or Cdc7.

16. A method according to claim 5, wherein said disorder or condition is selected from the group consisting of carcinoma, squamous cell carcinoma, hematopoietic tumors of myeloid or lymphoid lineage, tumors of mesenchymal origin, tumors of the central and peripheral nervous system, melanoma, seminoma, teratocarcinoma, osteosarcoma, xeroderma pigmentosum, keratoxanthoma, thyroid follicular cancer, and Kaposi's sarcoma.

17. A method according to claim 5, wherein said disorder or condition is selected from the group consisting of benign prostate hyperplasia, familial adenomatosis, polyposis, neuro-fibromatosis, psoriasis, vascular smooth cell proliferation associated with atherosclerosis, pulmonary fibrosis, arthritis glomerulonephritis, post-surgical stenosis and restenosis.

18. A method of treating a disorder or condition selected from the group consisting of squamous cell carcinoma, leukemia, acute lymphocitic leukemia, acute lymphoblastic leukemia, B-cell lymphoma, T-cell-lymphoma, Hodgkin's lymphoma, non-Hodgkin's lymphoma, hairy cell lymphoma and Burkitt's lymphoma, acute and chronic myelogenous leukemias, myelodysplastic syndrome, promyelocytic leukemia, fibrosarcoma, rhabdomyosarcoma, astrocytoma, neuroblastoma, glioma, schwannomas, melanoma, seminoma, teratocarcinoma, osteosarcoma, xeroderma pigmentosum, keratoxanthoma, thyroid follicular cancer, Kaposi's sarcoma and carcinoma of the bladder, breast, colon, kidney, liver, lung, esophagus, gall-bladder, ovary, pancreas, stomach, cervix, thyroid, prostate, or skin, in a mammal, comprising administering to said mammal in need of said treatment an amount of a compound of claim 1 that is effective in treating said condition or disorder.

19. A method of treating a disorder or condition selected from the group consisting of squamous cell carcinoma, leukemia, acute lymphocitic leukemia, acute lymphoblastic leukemia, B-cell lymphoma, T-cell-lymphoma, Hodgkin's lymphoma, non-Hodgkin's lymphoma, hairy cell lymphoma and Burkitt's lymphoma, acute and chronic myelogenous leukemias, myelodysplastic syndrome, promyelocytic leukemia, fibrosarcoma, rhabdomyosarcoma, astrocytoma, neuroblastoma, glioma, schwannomas, melanoma, seminoma, teratocarcinoma, osteosarcoma, xeroderma pigmentosum, keratoxanthoma, thyroid follicular cancer, Kaposi's sarcoma and carcinoma of the bladder, breast, colon, kidney, liver, lung, esophagus, gall-bladder, ovary, pancreas, stomach, cervix, thyroid, prostate, or skin, in a mammal, comprising administering to said mammal in need of said treatment an amount of a compound of claim 1 that is effective in antagonizing activity toward Cdk2 or Cdc7.

20. A method according to claim 5, wherein said disorder or condition is selected from the group consisting of squamous cell carcinoma, leukemia, acute lymphocitic leukemia, acute lymphoblastic leukemia, B-cell lymphoma, T-cell-lymphoma, Hodgkin's lymphoma, non-Hodgkin's lymphoma, hairy cell lymphoma and Burkitt's lymphoma, acute and chronic myelogenous leukemias, myelodysplastic syndrome, promyelocytic leukemia, fibrosarcoma, rhabdomyosarcoma, astrocytoma, neuroblastoma, glioma, schwannomas, melanoma, seminoma, teratocarcinoma, osteosarcoma, xeroderma pigmentosum, keratoxanthoma, thyroid follicular cancer, Kaposi's sarcoma and carcinoma of the bladder, breast, colon, kidney, liver, lung, esophagus, gall-bladder, ovary, pancreas, stomach, cervix, thyroid, prostate, or skin.

21. A pharmaceutical composition comprising an amount of the compound of claim 1, or a pharmaceutically acceptable salt or solvate thereof, and a pharmaceutically acceptable carrier.

22. A compound according to claim 1 selected from the group consisting of:
2-(2-Amino-pyrimidin-4-yl)-1-benzyl-1,5,6,7-tetrahydro-pyrrolo[3,2-c]pyridin-4-one;
2-Pyridin-4-yl-1-[2-(tetrahydro-pyran-2-yloxy)-ethyl]-1,5,6,7-tetrahydro-pyrrolo[3,2-c]pyridin-4-one;
1-(2-Hydroxy-ethyl)-2-pyridin-4-yl-1,5,6,7-tetrahydro-yrrolo[3,2-c]pyridin-4-one;
2-(4-Oxo-2-pyridin-4-yl-4,5,6,7-tetrahydro-pyrrolo[3,2-c]pyridin-1-yl)-acetamide;
4-Oxo-2-pyridin-4-yl-4,5,6,7-tetrahydro-pyrrolo[3,2-c]pyridine-1-carboxylic acid methyl ester;
1-Ethyl-2-pyridin-4-yl-1,5,6,7-tetrahydro-pyrrolo[3,2-c]pyridin-4-one;
2-Pyridin-4-yl-1-(2,2,2-trifluoro-ethyl)-1,5,6,7-tetrahydro-pyrrolo[3,2-c]pyridin-4-one;
2-(3-Fluoro-pyridin-4-yl)-1-(2,2,2-trifluoro-ethyl)-1,5,6,7-tetrahydro-pyrrolo[3,2-c]pyridin-4-one;
3-Methyl-2-pyridin-4-yl-1-(2,2,2-trifluoro-ethyl)-1,5,6,7-tetrahydro-pyrrolo[3,2-c]pyridin-4-one;
2-(2-Amino-pyrimidin-4-yl)-1-methyl-1,5,6,7-tetrahydro-pyrrolo[3,2-c]pyridin-4-one;
2-(2-Amino-pyrimidin-4-yl)-1-(2,2,2-trifluoro-ethyl)-1,5,6,7-tetrahydro-pyrrolo[3,2-c]pyridin-4-one;
2-(2-Amino-pyrimidin-4-yl)-1-ethyl-1,5,6,7-tetrahydro-pyrrolo[3,2-c]pyridin-4-one;
2-(2-Amino-pyrimidin-4-yl)-1-(4,4,4-trifluoro-butyl)-1,5,6,7-tetrahydro-pyrrolo[3,2-c]pyridin-4-one;
2-(2-Amino-pyrimidin-4-yl)-1-(4-trifluoromethyl-benzyl)-1,5,6,7-tetrahydro-pyrrolo[3,2-c]pyridin-4-one;
2-(2-Amino-pyrimidin-4-yl)-1-propyl-1,5,6,7-tetrahydro-pyrrolo[3,2-c]pyridin-4-one;
2-(2-Amino-pyrimidin-4-yl)-1-butyl-1,5,6,7-tetrahydro-pyrrolo[3,2-c]pyridin-4-one;
2-(2-Amino-pyrimidin-4-yl)-1-isobutyl-1,5,6,7-tetrahydro-pyrrolo[3,2-c]pyridin-4-one;
2-(2-Amino-pyrimidin-4-yl)-1-(2-hydroxy-ethyl)-1,5,6,7-tetrahydro-pyrrolo[3,2-c]pyridin-4-one;
2-(2-Amino-pyrimidin-4-yl)-6-benzyl-1-ethyl-1,5,6,7-tetrahydro-pyrrolo[3,2-c]pyridin-4-one;
2-(2-Amino-pyrimidin-4-yl)-1,7-diethyl-1,5,6,7-tetrahydro-pyrrolo[3,2-c]pyridin-4-one;
(R and S)-2-(2-Amino-pyrimidin-4-yl)-7-ethyl-1-(2,2,2-trifluoro-ethyl)-1,5,6,7-tetrahydro-pyrrolo[3,2-c]pyridin-4-one;
2-(2-Amino-pyrimidin-4-yl)-1-(2-fluoro-ethyl)-1,5,6,7-tetrahydro-pyrrolo[3,2-c]pyridin-4-one;
2-(2-Amino-pyrimidin-4-yl)-1-(2,2,2-trifluoro-ethyl)-1,5,6,7-tetrahydro-pyrrolo[3,2-c]pyridin-4-one;
2-(2-Amino-pyrimidin-4-yl)-6-isobutyl-1-(2,2,2-trifluoro-ethyl)-1,5,6,7-tetrahydro-pyrrolo[3,2-c]pyridin-4-one;
2-(2-Amino-pyrimidin-4-yl)-1-isopropyl-1,5,6,7-tetrahydro-pyrrolo[3,2-c]pyridin-4-one;
2-(2-Amino-pyrimidin-4-yl)-6,6-dimethyl-1-(2,2,2-trifluoro-ethyl)-1,5,6,7-tetrahydro-pyrrolo[3,2-c]pyridin-4-one;
2-(2-Amino-pyrimidin-4-yl)-7-methyl-1-(2,2,2-trifluoro-ethyl)-1,5,6,7-tetrahydro-pyrrolo[3,2-c]pyridin-4-one;
(R and S)-2-(2-Amino-pyrimidin-4-yl)-7-isopropyl-1-(2,2,2-trifluoro-ethyl)-1,5,6,7-tetrahydro-pyrrolo[3,2-c]pyridin-4-one;
2-(2-Amino-pyrimidin-4-yl)-1-cyclopropylmethyl-1,5,6,7-tetrahydro-pyrrolo[3,2-c]pyridin-4-one;
1-Methyl-2-pyridin-4-yl-1,5,6,7-tetrahydro-pyrrolo[3,2-c]pyridin-4-one;
2-(2-Amino-pyrimidin-4-yl)-1-phenyl-1,5,6,7-tetrahydro-pyrrolo[3,2-c]pyridin-4-one;
2-(2-Amino-pyrimidin-4-yl)-1-cyclopropyl-1,5,6,7-tetrahydro-pyrrolo[3,2-c]pyridin-4-one;
2-(2-Amino-pyrimidin-4-yl)-1-(1-methyl-piperidin-4-yl)-1,5,6,7-tetrahydro-pyrrolo[3,2-c]pyridin-4-one
2-(2-Amino-pyrimidin-4-yl)-1-cyclohexyl-1,5,6,7-tetrahydro-pyrrolo[3,2-c]pyridin-4-one;
2-(2-Amino-pyrimidin-4-yl)-7,7-dimethyl-1-(2,2,2-trifluoro-ethyl)-1,5,6,7-tetrahydro-pyrrolo[3,2-c]pyridin-4-one;
2-(2-Amino-pyrimidin-4-yl)-1-cyclopropylmethyl-7,7-dimethyl-1,5,6,7-tetrahydro-pyrrolo[3,2-c]pyridin-4-one;
2-(2-Amino-pyrimidin-4-yl)-1-cyclobutyl-1,5,6,7-tetrahydro-pyrrolo[3,2-c]pyridin-4-one;
2-(2-Amino-pyrimidin-4-yl )-6-benzyl-1-(2,2,2-trifluoro-ethyl)-1,5,6,7-tetrahydro-pyrrolo[3,2-c]pyridin-4-one;
2-(2-Amino-pyrimidin-4-yl)-1-(1-phenyl-ethyl)-1,5,6,7-tetrahydro-pyrrolo[3,2-c]pyridin-4-one;
2-(2-Amino-pyrimidin-4-yl)-7-cyclobutyl-1-ethyl-1,5,6,7-tetrahydro-pyrrolo[3,2-c]pyridin-4-one;
2-(2-Amino-pyrimidin-4-yl)-7-cyclobutyl-1-(2,2,2-trifluoro-ethyl)-1,5,6,7-tetrahydro-pyrrolo[3,2-c]pyridin-4-one;
2-(2-Amino-pyrimidin-4-yl)-7-cyclobutyl-1-propyl-1,5,6,7-tetrahydro-pyrrolo[3,2-c]pyridin-4-one;
2-(2-Amino-pyrimidin-4-yl)-7-cyclobutyl-1-isobutyl-1,5,6,7-tetrahydro-pyrrolo[3,2-c]pyridin-4-one;
2-(2-Amino-pyrimidin-4-yl)-7-cyclobutyl-1-cyclopropylmethyl-1,5,6,7-tetrahydro-pyrrolo[3,2-c]pyridin-4-one;
2-(2-Amino-pyrimidin-4-yl)-1,7-dicyclobutyl-1,5,6,7-tetrahydro-pyrrolo[3,2-c]pyridin-4-one;
2-(2-Amino-pyrimidin-4-yl)-7-cyclobutyl-1-cyclobutylmethyl-1,5,6,7-tetrahydro-pyrrolo[3,2-c]pyridin-4-one;
2-(2-Amino-pyrimidin-4-yl)-3-iodo-1-methyl-1,5,6,7-tetrahydro-pyrrolo[3,2-c]pyridin-4-one;
2-(2-Amino-pyrimidin-4-yl)-6-cyclopropyl-1-ethyl-1,5,6,7-tetrahydro-pyrrolo[3,2-c]pyridin-4-one;
2-(2-Amino-pyrimidin-4-yl)-6-cyclopropyl-1-(2,2,2-trifluoro-ethyl)-1,5,6,7-tetrahydro-pyrrolo[3,2-c]pyridin-4-one;
2-(2-Amino-pyrimidin-4-yl)-6-cyclopropyl-1-propyl-1,5,6,7-tetrahydro-pyrrolo[3,2-c]pyridin-4-one;
2-(2-Amino-pyrimidin-4-yl)-6-cyclopropyl-1-isobutyl-1,5,6,7-tetrahydro-pyrrolo[3,2-c]pyridin-4-one;
2-(2-Amino-pyrimidin-4-yl)-6-cyclopropyl-1-cyclopropylmethyl-1,5,6,7-tetrahydro-pyrrolo[3,2-c]pyridin-4-one;
2-(2-Amino-pyrimidin-4-yl)-1-cyclobutyl-6-cyclopropyl-1,5,6,7-tetrahydro-pyrrolo[3,2-c]pyridin-4-one; and
2-(2-Amino-pyrimidin-4-yl)-1-cyclobutylmethyl-6-cyclopropyl-1,5,6,7-tetrahydro-pyrrolo[3,2-c]pyridin-4-one.
